(19) United States
(12) Patent Application Publication
(10) Pub. No.: US 2023/0321012 A1
(43) Pub. Date: Oct. 12, 2023

(54) Methods To Treat Cancer With AMXT 1501 and DFMO

(71) Applicant: Aminex Therapeutics, Inc., Kenmore, WA (US)

(72) Inventors: Mark R. Burns, Epsom, NH (US); James Skaggs, Austin, TX (US)

(21) Appl. No.: 18/060,108

(22) Filed: Nov. 30, 2022

Related U.S. Application Data
(60) Provisional application No. 63/371,412, filed on Aug. 15, 2022.
(60) Provisional application No. 63/264,724, filed on Dec. 1, 2021.

(51) Int. Cl. A61K 31/16; A61K 31/198; A61K 9/00; A61P 35/00

(52) U.S. Cl. CPC A61K 31/16; A61K 31/198; A61K 9/0019; A61P 35/00

(57) ABSTRACT

Provided herein are methods of treating cancer, comprising administering AMXT 1501, or a pharmaceutically acceptable salt thereof, orally, and difluoromethylornithine (DFMO), or a pharmaceutically acceptable salt thereof, intravenously to a subject in need thereof. AMXT 1501 has the following structural formula:

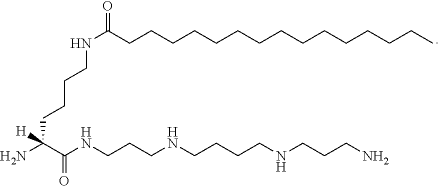 [SMILES: CCCCCCCCCCCCCCCC(=O)NCCCC[C@@H](N)C(=O)NCCCNCCCCNCCCN]

### RELATED APPLICATIONS

This application claims the benefit of U.S. Provisional Application No. 63/264,724, filed on Dec. 1, 2021, and U.S. Provisional Application No. 63/371,412, filed on Aug. 15, 2022. The entire teachings of the above applications are incorporated herein by reference.

### BACKGROUND

Nonclinical efficacy studies in mouse cancer models for both adult and pediatric tumor indications have demonstrated pronounced anti-tumor efficacy for AMXT 1501 plus difluoromethylornithine (DFMO). Targeting of tumor polyamine metabolism using AMXT 1501 plus DFMO in immune-competent animals has revealed an immunomodulatory effect of treatment, in addition to the potent anti-proliferative effects of this polyamine antimetabolite approach. Measurement of tumor polyamine reduction in AMXT 1501 plus DFMO-treated animals indicates the observed anti-tumor activities are mediated by sustained reduction of polyamine metabolites.

Clinical testing of cancer therapeutics that target polyamine metabolism have been limited by less than sustained, robust and durable engagement of polyamine-related biological targets. For instance, human testing of the ornithine decarboxylase (ODC) inhibitor DFMO against cancer is challenged by the rapid biological turnover of this enzyme. ODC's biological half-life is shorter than 10 minutes. Additionally, cells can overcome blockade of this rate-limiting enzyme in the polyamine biosynthetic pathway by increasing uptake of polyamines via a specific, and energy-dependent polyamine transporter. Except for some recent activity in the cancer chemoprevention setting, DFMO as a single agent has failed to show activity in the oncology clinic.

For AMXT 1501 plus DFMO, whose mode of action is dependent upon sustained suppression of tumor or tumor microenvironmental polyamine levels, it is especially important to achieve sustained and durable target engagement. This drug-target engagement is heavily influenced by the schedule and mode of delivery in preclinical animal and human clinical settings.

Although combination drug therapy has a robust history for several important clinical applications, including antibiotic treatments, antiviral treatments and oncology therapeutics, combination drug therapy involving two drugs that target the same biological pathway is rarer. AMXT 1501 and DFMO both target the polyamine metabolic pathway for the treatment of cancer. The challenging nature of dual agent dose-escalation against a single metabolic pathway (polyamines) in the clinical setting requires greater insight and management of drug delivery methods and pharmacokinetic levels of the drugs, together with a fuller evaluation of drug target engagement. This information is needed not only for patient safety, but also for estimations of drug levels required for optimal patient benefit.

Accordingly, there is a need for methods of delivering AMXT 1501 and DFMO to a cancer patient to provide a combination drug therapy that is safe and efficacious.

### SUMMARY

One embodiment provides a method of treating cancer in a subject in need thereof, comprising administering to the subject a therapeutically effective amount of AMXT 1501, or a pharmaceutically acceptable salt thereof (e.g., AMXT 1501 dicaprate), and difluoromethylornithine (DFMO), or a pharmaceutically acceptable salt thereof, wherein the AMXT 1501, or a pharmaceutically acceptable salt thereof, is administered orally, and the DFMO, or a pharmaceutically acceptable salt thereof, is administered intravenously.

Another embodiment provides a method of treating cancer in a subject in need thereof, comprising administering to the subject a therapeutically effective amount of AMXT 1501, or a pharmaceutically acceptable salt thereof, and DFMO, or a pharmaceutically acceptable salt thereof, wherein the AMXT 1501, or a pharmaceutically acceptable salt thereof, is administered orally QD or BID at a daily dose of greater than 80 mg and less than 2,500 mg, based on AMXT 1501.

Also provided herein is a pharmaceutical combination comprising AMXT 1501, or a pharmaceutically acceptable salt thereof (e.g., AMXT 1501 dicaprate), and DFMO, or a pharmaceutically acceptable salt thereof, for use in treating cancer. Also provided herein is use of a pharmaceutical combination comprising AMXT 1501, or a pharmaceutically acceptable salt thereof (e.g., AMXT 1501 dicaprate), and DFMO, or a pharmaceutically acceptable salt thereof, for the manufacture of a medicament for the treatment of cancer. In some embodiments, AMXT 1501, or a pharmaceutically acceptable salt thereof, is orally administrable, and DFMO, or a pharmaceutically acceptable salt thereof, is intravenously administrable. In some embodiments, AMXT 1501, or a pharmaceutically acceptable salt thereof, is orally administrable QD or BID at a daily dose of greater than 80 mg and less than 2,500 mg, based on AMXT 1501.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments.

FIG. 1A is a mean plasma concentration versus time profile, and shows the mean plasma concentration of AMXT 1501 after a single dose (Day 1).

FIG. 1B is a mean plasma concentration versus time profile, and shows the mean plasma concentration of AMXT 1501 after repeat dosing (Day 14).

FIG. 2 is a mean plasma concentration versus time profile, and shows the mean plasma concentration of DFMO after repeat dosing of 500 mg daily, split BID (Day 28).

FIG. 3A shows percent inhibition of C5 uptake in Day 7-8 (D7-D8) ex vivo patient blood CD4+ and CD4− lymphocytes from Part 2 Cohort 3 Patient (Pt) 3001-0008. Percent inhibition is relative to Day 1 pretreatment (0 hour) sample uptake of 100% (dashed lines).

FIG. 3B shows percent inhibition of C5 uptake in Day 7-8 ex vivo patient blood CD4+ and CD4− lymphocytes from Part 2 Cohort 3 Pt 3001-0009. Percent inhibition is relative to Day 1 pretreatment (0 hour) sample uptake of 100% (dashed lines).

FIG. 3C shows percent inhibition of C5 uptake in Day 7-8 ex vivo patient blood CD4+ and CD4− lymphocytes from Part 2 Cohort 3 Pt 1101-0001. Percent inhibition is relative to Day 1 pretreatment (0 hour) sample uptake of 100% (dashed lines).

FIG. 4A shows tumor pharmacokinetics and retention of AMXT 1501 with and without DFMO co-treatment (conditions: mean (±SD) AMXT 1501 plasma concentrations (ng/mL) and tumor concentrations (ng/g) after a single, SC dose of 2.5 mg/kg AMXT 1501 4HCl with or without PO DFMO pre-treatment (400 mg/kg for 3 days) in 4T1 breast tumor-bearing (7 days after injection of 1×10⁵ 4T1 tumor cells) mice). Average shown for n=3 mice at each time point.

FIG. 4B shows mouse plasma, tumor and tissue distribution of AMXT 1501 following a single subcutaneous injection (2.5 mg/kg) with 3-day DFMO (400 mg/kg) pretreatment (conditions: mean (±SD) AMXT 1501 plasma concentrations (ng/mL), tissues and tumor concentrations (ng/g) in 4T1 tumor-bearing mice (7 days after injection of 1×10⁵ 4T1 tumor cells) after a single, 2.5 mg/kg AMXT 1501 4HCl SC dose following 3 days pre-treatment with 400 mg/kg DFMO PO daily). Average shown for n=3 mice at each time point.

FIG. 5A shows the gating strategy for the acquisition of patient samples described in Example 4. Please note the difference in the scatter properties of cells between the negative control (top row) and samples containing anti-CD3/anti-CD28 (bottom row). The lymphocyte and live CD3 gate required adjustment to include proliferating T cells.

FIG. 5B shows titration of AMXT 1501 for inhibition of C5 uptake into CD4 lymphocytes from whole blood.

FIG. 5C shows titration of AMXT 1501 for inhibition of C5 uptake into CD8 lymphocytes from whole blood.

FIG. 5D shows summary data for AMXT 1501 inhibition of C5 uptake into CD4 lymphocytes.

FIG. 5E shows summary data for AMXT 1501 inhibition of C5 uptake into CD8 lymphocytes.

FIG. 6 is a concentration versus time profile, and shows AMXT 1501 biopsy and plasma PK data from a melanoma patient.

### DETAILED DESCRIPTION

A description of example embodiments follows.

### Definitions

As used in this specification and the appended claims, the singular forms “a,” “an,” and “the” include plural referents, unless the content and context clearly dictates otherwise.

Unless the context requires otherwise, throughout the specification and claims that follow, the word “comprise” and synonyms and variants thereof such as “have” and “include”, as well as variations thereof, such as “comprises” and “comprising”, are to be construed in an open, inclusive sense, e.g., “including, but not limited to.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, ±10%, ±5% or ±1% of a given value. It is to be understood that the term “about” can precede any particular value specified herein, except for particular values used in the Examples.

The term “salt” has its standard meaning in the art, and refers to a positively charged species (cation) and a negatively charged species (anion) that are complexed to one another through an ionic interaction. Generally, these salts do not involve covalent bonding between partner molecular components. Salts can be obtained by customary methods known to those skilled in the art, for example, by combining a compound with an inorganic or organic acid or base in a solvent or diluent, or from other salts by cation exchange or anion exchange.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

The term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. An example list of pharmaceutically acceptable salts can be found in the Handbook of Pharmaceutical Salts: Properties, Selection and Use, P. H. Stahl and C. G. Wermuth editors, Weinheim/Zurich:Wiley-VCHA/VHCA, 2002, the relevant teachings of which are incorporated herein by reference in their entirety. Pharmaceutically acceptable salts include pharmaceutically acceptable acid addition salts and pharmaceutically acceptable base addition salts.

Examples of pharmaceutically acceptable acid addition salts are salts of an amino group formed with inorganic acids such as hydrochloric acid, hydrobromic acid, phosphoric acid, sulfuric acid and perchloric acid, or with organic acids, such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like. Either the mono-, di-, tri or tetra-acid salts can be formed, and such salts can exist in either a hydrated, solvated or substantially anhydrous form.

Other pharmaceutically acceptable acid addition salts include salts formed with hydrophobic carboxylic acids, such as those described in U.S. Pat. No. 10,632,145, the entire content of which is incorporated herein by reference in its entirety. Hydrophobic carboxylic acids include those carboxylic acid-containing compounds having a water solubility of less than 10 g/L, e.g., less than 1 g/L, or less than 0.1 g/L, or less than 0.01 g/L in water, as determined at a temperature of 25° C. and a pH of 7. Compendiums of the water solubility of carboxylic acid-containing compounds may be found in, e.g., Yalkowsky S H, Dannenfelser R M; The AQUASOL database of Aqueous Solubility. Fifth ed., Tucson, Ariz.: Univ. AZ, College of Pharmacy (1992); Yalkowsky S H et al; Arizona Data Base of Water Solubility (1989); and The Handbook of Aqueous Solubility Data, Second Edition, edited by Yalkowsky S H, He, Y, and Jain, P, CRC Press (2010). Specific examples of hydrophobic carboxylic acids include fatty acids, such as C₈-C₁₈ straight chain hydrocarbon fatty acids, such as octanoic acid (also known as caprylic acid), nonanoic acid, decanoic acid (also known as capric acid), undecanoic acid, dodecanoic acid (also known as lauric acid), tridecanoic acid, tetradecanoic acid and hexadecanoic acid. Capric acid is a preferred hydrophobic carboxylic acid.

Pharmaceutically acceptable base addition salts include salts formed with inorganic bases, such as alkali metal, alkaline earth metal, and ammonium bases, and salts formed with aliphatic, alicyclic or aromatic organic amines, such as methylamine, trimethylamine and picoline, or N+((C₁-C₄)alkyl)₄ salts. Representative alkali or alkaline earth metal salts include sodium, lithium, potassium, calcium, magnesium, barium and the like. Further pharmaceutically acceptable base addition salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared.

Compounds may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers (including cis and trans double bond isomers), conformational isomers (including rotamers and atropisomers), tautomers, and intermediate mixtures, with all possible isomers and mixtures thereof being included, unless otherwise indicated.

When a disclosed compound is depicted by structure or named without indicating the stereochemistry, and the compound has one or more chiral centers, it is to be understood that the structure encompasses one enantiomer or diastereomer of the compound separated or substantially separated from the corresponding optical isomer(s), a racemic mixture of the compound, and mixtures enriched in one enantiomer or diastereomer relative to its corresponding optical isomer(s). When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” can be used to indicate the absolute configuration of substituents around one or more chiral carbon atoms. D- and L-can also be used to designate stereochemistry.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D4 . . . ) represent the respective fractions of each diastereomer in a mixture, such that D1+(D2+D3+D4 . . . )=1. A diastereomer may be present in a de of at least or about 50%, about 60%, about 70%, about 80%, about 90%, about 95%, about 98%, about 99% or about 99.9%.

Compounds can also exist as isotopologues, differing from a disclosed structure only in the presence of one or more isotopically enriched atoms. For example, compounds produced by the replacement of a hydrogen with deuterium or tritium, or of a carbon with a ¹³C- or 14C-enriched carbon are within the scope of this disclosure. In all provided structures, any hydrogen atom can also be independently selected from deuterium (²H), tritium (3H) and/or fluorine (¹⁸F). Such compounds are useful, for example, as analytical tools, as probes in biological assays, or as therapeutic agents in accordance with the present disclosure.

When a therapeutically active agent (e.g., AMXT 1501, DFMO) is referred to herein, unless the context indicates otherwise, the reference includes reference to the recited agent, as well as any isomers, such as stereoisomers (including diastereoisomers, enantiomers and racemates) and tautomers, isotopologues, inherently formed moieties (e.g., polymorphs and/or solvates, such as hydrates), ionic forms or salts (e.g., pharmaceutically acceptable salts) thereof. Reference to DFMO, or a pharmaceutically acceptable salt thereof, includes solvates and/or hydrates of DFMO or the pharmaceutically acceptable salt thereof, such as DFMO monohydrochloride monohydrate.

“Pharmaceutically acceptable carrier” refers to a non-toxic carrier or excipient that does not destroy the pharmacological activity of an agent(s) with which it is formulated and is nontoxic when administered in doses sufficient to deliver a therapeutic amount of the agent(s). Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treatment” and “treating” refer to medical management of a disease, disorder, or condition of a subject. A treatment may improve or decrease the severity at least one symptom of a disease, disorder or condition; delay worsening or progression of a disease, disorder or condition; delay or prevent onset of additional associated diseases, disorders or conditions; or improve remodeling of lesions into functional (partially or fully) tissue.

A “therapeutically effective amount” or “effective amount” of a therapy refers to that amount sufficient to result in amelioration of one or more symptoms of the disease being treated in a statistically significant manner. When referring to an individual active ingredient administered alone, a therapeutically effective amount refers to that ingredient alone. When referring to a combination, a therapeutically effective amount refers to combined amounts of the active ingredients that result in the therapeutic effect, whether administered serially or simultaneously.

“Subject,” as used herein, refers to a mammal. In addition to warm-blooded animals, such as mice, rats, horses, cattle, sheep, dogs, cats, monkeys, etc., “subject” includes humans. In some embodiments, a subject is a veterinary subject, such as a dog or cat (e.g., domestic cat). In preferred embodiments, a subject is a human (a “patient”). In some embodiments, the human is an adult human (adult patient). In some embodiments, the human is a pediatric human (pediatric patient, e.g., aged twelve to seventeen years-old). In some embodiments, the human is an adult human or a human aged 12- to 17-years old. Pediatric patients younger than 12 years old may also be treated by the methods described herein.

“Subject in need” refers to a subject at risk of, or suffering from, a disease, disorder or condition that is amenable to treatment in accordance with the present disclosure. In certain embodiments, a subject in need is a mammal, e.g., a human, such as an adult or pediatric human.

### Methods

Provided herein is a method of treating cancer in a subject (e.g., a subject in need thereof), comprising administering to the subject AMXT 1501, or a pharmaceutically acceptable salt thereof, and DFMO, or a pharmaceutically acceptable salt thereof (e.g., a therapeutically effective amount of AMXT 1501, or a pharmaceutically acceptable salt thereof, DFMO, or a pharmaceutically acceptable salt thereof).

AMXT 1501 is D-lys(palmitoyl)-spermine, and has the following structure:

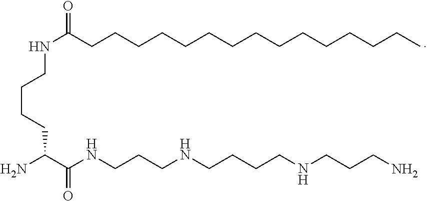 [SMILES: CCCCCCCCCCCCCCCC(=O)NCCCC[C@@H](N)C(=O)NCCCNCCCCNCCCN]

In some embodiments, AMXT 1501, or a pharmaceutically acceptable salt thereof, is AMXT 1501 dicaprate. In some embodiments, the AMXT 1501 dicaprate has the following structure:

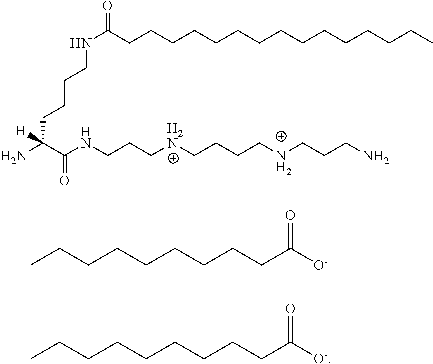

AMXT 1501 tetrahydrochloride is available from Aminex Therapeutics (Epsom, NH, USA). Methods of making the free base form of AMXT 1501, as well as salts thereof, including AMXT 1501 dicaprate, can be found, for example, in International Publication No. WO 2017/165313, the entire content of which is incorporated herein by reference.

DFMO has the following structure:

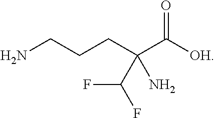 [SMILES: NCCCC(N)(C(=O)O)C(F)F]

It has been shown that the L-enantiomer of DFMO is a more potent inhibitor of ODC than is the D-enantiomer. Qu, N., et al., Biochem. J. (2003) 375, 465-470, the entire content of which is incorporated herein by reference. Accordingly, in some embodiments, DFMO is L-DFMO. L-DFMO has the following structure:

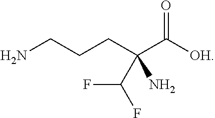 [SMILES: NCCC[C@](N)(C(=O)O)C(F)F]

In some embodiments, DFMO is D-DFMO. D-DFMO has the following structure:

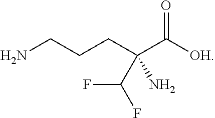 [SMILES: NCCC[C@@](N)(C(=O)O)C(F)F]

In some embodiments, DFMO is racemic.

DFMO and L-DFMO are commercially available, for example, from Tocris (Catalog No. 2761) and MedChemExpress (Catalog No. HY-B0744C), respectively. Methods of making DFMO, as well as its individual enantiomers, are also described in Qu, N., et al., Biochem. J. (2003) 375, 465-470; U.S. Pat. No. 4,330,559; Boberg, M. et al., ACS Omega 2020, 5, 23885-23891; and Peng, L. et al., J. Am. Chem. Soc. 2021, 143, 6376-6381, the entire contents of which are incorporated herein by reference.

The compounds and compositions described herein (e.g., AMXT 1501, or a pharmaceutically acceptable salt thereof, and/or DFMO, or a pharmaceutically acceptable salt thereof) can be administered by any route, with any frequency, and in any amount described herein. For example, the compounds and compositions described herein may be administered by oral, parenteral (e.g., intramuscular, intraperitoneal, intravenous, intracerebroventricular, intracisternal injection or infusion, subcutaneous injection, or implant), inhalation spray, nasal, vaginal, rectal, sublingual, or topical routes of administration.

In some embodiments, the AMXT 1501, or a pharmaceutically acceptable salt thereof, is administered orally (e.g., BID). In some embodiments, the DFMO, or a pharmaceutically acceptable salt thereof, is administered orally (e.g., BID), for example, to treat colorectal cancer. In some embodiments, the DFMO, or a pharmaceutically acceptable salt thereof, is administered by inhalation (e.g., via an inhaler), for example, to treat lung cancer. Methods and technologies to administer DFMO via aerosol inhalation are provided, for example, in Liao et al., LUNG DISTRIBUTION OF THE CHEMOPREVENTIVE AGENT DIFLUOROMETHYLORNITHINE (DFMO) FOLLOWING ORAL AND INHALATION DELIVERY. Exp. Lung Res. 2004, 30, 755-569. See also Watterberg, L. W., et. al., Cancer Res. 2004, 64 (7), 2347-9. In preferred embodiments, the DFMO is administered intravenously (e.g., by continuous intravenous infusion). Thus, in some preferred embodiments, the AMXT 1501, or a pharmaceutically acceptable salt thereof, is administered orally (e.g., BID), and the DFMO, or a pharmaceutically acceptable salt thereof, is administered intravenously (e.g., by continuous intravenous infusion).

The compounds and compositions described herein (e.g., AMXT 1501, or a pharmaceutically acceptable salt thereof, and/or DFMO, or a pharmaceutically acceptable salt thereof) may be administered as a single daily dose or in divided doses (e.g., two to six times per day), or as a continuous infusion. The compounds and compositions described herein may be administered on a regimen of 1 to 4 times per day, preferably once or twice per day (e.g., QD or BID), e.g., at the discretion of the attending health care professional.

In some embodiments, AMXT 1501, or a pharmaceutically acceptable salt thereof, is administered once daily (QD) or twice daily (BID). In some embodiments, AMXT 1501, or a pharmaceutically acceptable salt thereof, is administered once daily (QD). In preferred embodiments, AMXT 1501, or a pharmaceutically acceptable salt thereof, is administered twice daily (BID). Thus, in some preferred embodiments, a recited daily dose of AMXT 1501, or a pharmaceutically acceptable salt thereof, is split BID. When it is stated that a recited daily dose is split BID, it will be understood that the recited dose is divided into two portions (e.g., typically, equal or approximately equal portions) that are administered at different times of day (e.g., typically, in the morning and in the evening).

In some embodiments, AMXT 1501, or a pharmaceutically acceptable salt thereof, is administered to a subject in a fasted state. In some embodiments, AMXT 1501, or a pharmaceutically acceptable salt thereof, is administered to a subject in a fed state.

In preferred embodiments, DFMO, or a pharmaceutically acceptable salt thereof, is administered by continuous infusion (e.g., continuous intravenous infusion), for example, via an ambulatory infusion system, such as an ambulatory pump.

Ambulatory infusion systems, such as elastomeric pumps, enable patients to receive an intravenous infusion (e.g., continuous intravenous infusion) of a therapeutically active agent, such as DFMO, at home or work, rather than in a hospital or other medical setting. An especially appealing and straightforward technology is the elastomer drug infusion pumps offered by Avanos Medical Devices (sold under the name trade name Homepump, e.g., Homepump C-series). The Avanos drug infusion pumps are routinely used for continuous infusions of many types of pharmaceuticals, including pain management agents, oncology agents and antibiotic therapies. No electronics are required. Typically, an elastic ‘ball’ is loaded with drug solution under aseptic conditions. Sterile tubing inserts in an infusion port (central catheter) on the patient. A valve in the line regulates the rate of drug infusion (2 mL/hr, or other rates). The system provides drug infusion for up to 5.5 days or longer, and can be easily stopped at any time. Patients may, or may not, require nursing assistance to change the pump with new drug solution after, e.g., 5 days. Pumps with various rates of infusion and drug volumes are available.

The compounds and compositions described herein (e.g., AMXT 1501, or a pharmaceutically acceptable salt thereof, and/or DFMO, or a pharmaceutically acceptable salt thereof) will be administered at an appropriate dosage level, typically, from about 0.01 mg to about 1,000 mg (e.g., from about 0.01 mg to about 500 mg) per kg patient body weight per day, which can be administered in single or multiple doses. Optionally, the dosage level will be from about 0.1 to about 250 mg/kg per day; or from about 0.5 to about 100 mg/kg per day. A suitable dosage level may be from about 0.01 to about 250 mg/kg per day, from about 0.05 to about 100 mg/kg per day, from about 0.1 to about 100 mg/kg per day, or from about 0.1 to about 50 mg/kg per day. Within this range the dosage may be, for example, from about 0.05 to about 0.5, from about 0.5 to about 5, from about 1 to about 50, or from about 5 to about 50 mg/kg per day. For most large mammals, the total daily dosage will typically be from about 1 milligram to about 10,000 milligrams, or from about 1 milligram to about 5,000 milligrams, or from about 1 milligram to about 2,500 milligrams, or from about 1 milligram to about 1,000 milligrams.

Human dose levels, especially those used for cancer chemotherapy, are alternatively expressed in units of mg/m²/day. For example, a daily dosage of a compound described herein (e.g., AMXT 1501, or a pharmaceutically acceptable salt thereof, and/or DFMO, or a pharmaceutically acceptable salt thereof) of from about 0.1 gram to about 100 grams per meter squared of body weight can be given. For most large mammals, a total daily dosage can be from about 1 gram to about 1,000 grams, or from about 1 gram to about 50 grams. Dosages may be adjusted to provide optimal therapeutic response.

In some embodiments, AMXT 1501, or a pharmaceutically acceptable salt thereof, is administered (e.g., orally) at a daily dose of from about 50 mg to about 5,000 mg, e.g., greater than 80 mg to less than 2,500 mg, from about 100 mg to about 2,500 mg, from about 100 mg to about 1,800 mg, about 800 mg, about 1,200 mg or about 1,800 mg, based on the free base of AMXT 1501. In some embodiments, the daily dose of AMXT 1501, or a pharmaceutically acceptable salt thereof, is split BID. In some embodiments, AMXT 1501, or a pharmaceutically acceptable salt thereof, is administered orally QD or BID (e.g., BID), at a daily dose of from about 50 mg to about 5,000 mg, e.g., greater than 80 mg to less than 2,500 mg, from about 100 mg to about 2,500 mg, from about 100 mg to about 1,800 mg, about 800 mg, about 1,200 mg or about 1,800 mg, based on the free base of AMXT 1501.

It has been found the oral delivery of 1,200 mg AMXT 1501 dicaprate (based on the free base of AMXT 1501) alone, either once daily or split BID, is well-tolerated clinically. In some embodiments, AMXT 1501, or a pharmaceutically acceptable salt thereof (e.g., AMXT 1501 dicaprate), is administered orally QD or BID (e.g., BID) at a daily dose of 1,200 mg, based on the free base of AMXT 1501.

In some embodiments, administration (e.g., oral administration) of AMXT 1501, or a pharmaceutically acceptable salt thereof, provides a therapeutically effective systemic plasma level of AMXT 1501 for a period of at least 4, 5, 6, 7, 8, 9, 10, 11, 12, 13, 14, 15, 16, 17, 18, 19, 20, 21, 22, 23, 24, 30, 36 or 48 hours, or 3, 4 or 5 days. In further embodiments, administration (e.g., oral administration) of AMXT 1501, or a pharmaceutically acceptable salt thereof, provides a therapeutically effective systemic plasma level of AMXT 1501 for at least an 8-hour period. In further embodiments, administration (e.g., oral administration) of AMXT 1501, or a pharmaceutically acceptable salt thereof, provides a therapeutically effective systemic plasma level of AMXT 1501 for at least a 12-hour period. In further embodiments, administration (e.g., oral administration) of AMXT 1501, or a pharmaceutically acceptable salt thereof, provides a therapeutically effective systemic plasma level of AMXT 1501 for at least a 14-hour period. In further embodiments, administration (e.g., oral administration) of AMXT 1501, or a pharmaceutically acceptable salt thereof, provides a therapeutically effective systemic plasma level of AMXT 1501 for at least an 18-hour period. In further embodiments, administration (e.g., oral administration) of AMXT 1501, or a pharmaceutically acceptable salt thereof, provides a therapeutically effective systemic plasma level of AMXT 1501 for at least a 24-hour period. In further embodiments, administration (e.g., oral administration) of AMXT 1501, or a pharmaceutically acceptable salt thereof, provides a therapeutically effective systemic plasma level of AMXT 1501 for at least a 3-day period. In further embodiments, administration (e.g., oral administration) of AMXT 1501, or a pharmaceutically acceptable salt thereof, provides a therapeutically effective systemic plasma level of AMXT 1501 for at least a 4-day period. In further embodiments, administration (e.g., oral administration) of AMXT 1501, or a pharmaceutically acceptable salt thereof, provides a therapeutically effective systemic plasma level of AMXT 1501 for at least a 5-day period.

In some embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for a period of at least 4, 5, 6, 7, 8, 9, 10, 11, 12, 13, 14, 15, 16, 17, 18, 19, 20, 21, 22, 23, 24, 30, 36 or 48 hours, or 3, 4 or 5 days. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least an 8-hour period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least a 12-hour period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least a 14-hour period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least an 18-hour period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least a 24-hour period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least a 3-day period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least a 4-day period. In further embodiments, administration (e.g., oral administration) of AMXT 1501, or a pharmaceutically acceptable salt thereof, provides a systemic plasma level of AMXT 1501 of greater than or about 50 nM (and, in further embodiments, greater than or about 100 nM) for at least a 5-day period.

In some embodiments, DFMO, or a pharmaceutically acceptable salt thereof, is administered (e.g., intravenously) to the subject at a daily dose of from about 0.1 g/m² to about 25 g/m², e.g., from about 0.5 g/m² to about 10 g/m², from about 1 g/m² to about 5 g/m², about 1 g/m², about 2 g/m², about 3 g/m² or about 4 g/m², based on the free base of DFMO, or alternatively, the DFMO monohydrochloride monohydrate salt. In some embodiments, the daily dose of DFMO, or a pharmaceutically acceptable salt thereof, is administered by continuous infusion (e.g., continuous intravenous infusion).

In some embodiments, DFMO, or a pharmaceutically acceptable salt thereof, is administered (e.g., orally) to the subject at a daily dose of from about 50 mg to about 25 g, e.g., from about 250 mg to about 5 g, from about 250 mg to about 2.5 g, about 250 mg, about 500 mg, about 1 g or about 2 g, based on the free base of DFMO. In some embodiments, the daily dose of DFMO, or a pharmaceutically acceptable salt thereof, is administered QD or split BID (e.g., split BID).

In some embodiments, administration (e.g., intravenous administration) of DFMO, or a pharmaceutically acceptable salt thereof, provides a therapeutically effective systemic plasma level of DFMO for a period of at least 4, 5, 6, 7, 8, 9, 10, 11, 12, 13, 14, 15, 16, 17, 18, 19, 20, 21, 22, 23, 24, 30, 36 or 48 hours, or 3, 4 or 5 days. In further embodiments, administration (e.g., intravenous administration) of DFMO, or a pharmaceutically acceptable salt thereof, provides a therapeutically effective systemic plasma level of DFMO for at least an 8-hour period. In further embodiments, administration (e.g., intravenous administration) of DFMO, or a pharmaceutically acceptable salt thereof, provides a therapeutically effective systemic plasma level of DFMO for at least a 12-hour period. In further embodiments, administration (e.g., intravenous administration) of DFMO, or a pharmaceutically acceptable salt thereof, provides a therapeutically effective systemic plasma level of DFMO for at least a 14-hour period. In further embodiments, administration (e.g., intravenous administration) of DFMO, or a pharmaceutically acceptable salt thereof, provides a therapeutically effective systemic plasma level of DFMO for at least an 18-hour period. In further embodiments, administration (e.g., intravenous administration) of DFMO, or a pharmaceutically acceptable salt thereof, provides a therapeutically effective systemic plasma level of DFMO for at least a 24-hour period. In further embodiments, administration (e.g., intravenous administration) of DFMO, or a pharmaceutically acceptable salt thereof, provides a therapeutically effective systemic plasma level of DFMO for at least a 3-day period. In further embodiments, administration (e.g., intravenous administration) of DFMO, or a pharmaceutically acceptable salt thereof, provides a therapeutically effective systemic plasma level of DFMO for at least a 4-day period. In further embodiments, administration (e.g., intravenous administration) of DFMO, or a pharmaceutically acceptable salt thereof, provides a therapeutically effective systemic plasma level of DFMO for at least a 5-day period.

In some embodiments, administration (e.g., intravenous administration) of DFMO, or a pharmaceutically acceptable salt thereof, provides a systemic plasma level of DFMO of greater than or about 39 μM (and, in further embodiments, greater than or about 100 PM) for a period of at least 4, 5, 6, 7, 8, 9, 10, 11, 12, 13, 14, 15, 16, 17, 18, 19, 20, 21, 22, 23, 24, 30, 36 or 48 hours, or 3, 4 or 5 days. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 μM) for at least an 8-hour period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 μM) for at least a 12-hour period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 PM) for at least a 14-hour period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 PM) for at least an 18-hour period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 μM) for at least a 24-hour period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 μM) for at least a 3-day period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 μM) for at least a 4-day period. In further embodiments, administration (e.g., intravenous administration) of DFMO, or a pharmaceutically acceptable salt thereof, provides a systemic plasma level of DFMO of greater than or about 39 μM (and, in further embodiments, greater than or about 100 μM) for at least a 5-day period.

It will be understood, however, that the specific dose level and frequency of dosage for any particular subject may be varied and will depend upon a variety of factors including the activity of the specific combination employed, the metabolic stability and length of action of the components of the combination, the age, body weight, body surface area, general health, sex, diet, mode and time of administration, rate of excretion, drug combination, the severity of the particular condition, and the subject undergoing therapy.

A variety of cancers are amenable to treatment in accordance with the present disclosure. In some embodiments, the cancer comprises a solid tumor. In some embodiments, the cancer is a solid tumor cancer. In some embodiments, the cancer is a hematological cancer, e.g., a leukemia, lymphoma or myeloma. The cancer may be, for example, breast cancer, prostate cancer, colon cancer or lung cancer. Other cancers that may be treated by appropriate selection of the polyamine transport inhibitor include neuroblastoma, pancreatic, bladder, melanoma, skin cancer, non-Hodgkin lymphoma, kidney cancer, head and neck cancers including glioblastoma, leukemia and other blood cancers, ovarian and thyroid cancers. The cancer may be treated by polyamine transport inhibitors that are specific for oncogenes, e.g., MYC- and/or RAS-derived tumors.

Other cancers amenable to treatment in accordance with the present disclosure include Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sézary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sézary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein. In some embodiments, the cancer is a metastatic cancer.

In some embodiments, the cancer is a pediatric cancer. “Pediatric cancer” typically refers to cancer that occurs between birth and fourteen years of age. Examples of pediatric cancers include neuroblastoma and diffuse intrinsic pontine glioma (DIPG).

In some embodiments, the cancer is skin cancer, melanoma, colon cancer, colorectal cancer, breast cancer, pancreatic cancer, neuroblastoma or DIPG. In some embodiments, the cancer is ovarian cancer, thyroid cancer, head and neck cancer, gastric cancer, lung cancer (e.g., non-small cell lung cancer, NSCLC), mesothelioma, esophageal cancer, endometrial cancer, cervical cancer, melanoma, DIPG (e.g., juvenile DIPG), colorectal cancer or breast cancer. In some embodiments, the cancer is colorectal, skin, head and neck, breast, bladder or pancreatic cancer, or DIPG (e.g., juvenile DIPG).

As described herein, high levels of AMXT 1501 retention in lung tissues have been observed (by LC/MS²) in 4T1 breast cancer tumor-bearing mice and (by MALDI) in neuroblastoma tumor-bearing mice. In some embodiments, the cancer is lung cancer. In some embodiments, the cancer has metastasized to the lung.

In general, there is a myriad of therapeutic uses for the combinations of the disclosure in addition to use as an anti-cancer treatment. For example, polyamine transport inhibitors, such as AMXT 1501, have been described to have antibiotic, antiviral, anti-inflammatory, anti-sepsis, anti-pain, anti-psychotic, anti-aging, and anti-heart damage activities, among others. Accordingly, also provided herein are methods of treating a disease, disorder or condition in a subject in need thereof, comprising administering to the subject a therapeutically effective amount of AMXT 1501, or a pharmaceutically acceptable salt thereof, and DFMO, or a pharmaceutically acceptable salt thereof, wherein the disease, disorder or condition would benefit from treatment with an agent having antibiotic, antiviral, anti-inflammatory, anti-sepsis, anti-pain, anti-psychotic, anti-aging and anti-heart damage activity.

In some embodiments, a method further comprises administering to a subject one or more additional therapies (e.g., therapeutically active agents), e.g., which are beneficially applied in the treatment of a disease, disorder or condition, such as cancer, experienced or potentially experienced by a subject receiving a combination therapy described herein.

Treatment of transgenic TH-MYCN mice bearing palpable neuroblastoma tumors with AMXT 1501 dicaprate, DFMO and standard of care agents cyclophosphamide and topotecan in the presence or absence of celecoxib has been shown to significantly prolong survival of the mice. Weiss, W. A., Aldape, K., Mohapatra, G., Feuerstein, B. G. & Bishop, J. M. Targeted expression of MYCN causes neuroblastoma in transgenic mice. EMBO J 16, 2985-2995 (1997). Accordingly, in some embodiments, the one or more additional therapies comprises or consists of a standard of care therapy, e.g., for neuroblastoma. In some embodiments, one or more additional therapeutically active agents comprises cyclophosphamide, topotecan, celecoxib, or a pharmaceutically acceptable salt thereof, or a combination of any of the foregoing (e.g., cyclophosphamide, or a pharmaceutically acceptable salt thereof, and topotecan, or a pharmaceutically acceptable salt thereof; cyclophosphamide, or a pharmaceutically acceptable salt thereof, and topotecan, or a pharmaceutically acceptable salt thereof, and celecoxib, or a pharmaceutically acceptable salt thereof).

When AMXT 1501, or a pharmaceutically acceptable salt thereof, and DFMO, or a pharmaceutically acceptable salt thereof, is administered with one or more additional therapies, AMXT 1501, or a pharmaceutically acceptable salt thereof, and/or DFMO, or a pharmaceutically acceptable salt thereof, can be administered before, after or concurrently with the other therapy(ies). When co-administered concurrently, AMXT 1501, or a pharmaceutically acceptable salt thereof, and/or DFMO, or a pharmaceutically acceptable salt thereof, and additional therapeutically active agent(s) can be in separate formulations or the same formulation. Alternatively, AMXT 1501, or a pharmaceutically acceptable salt thereof, and/or DFMO, or a pharmaceutically acceptable salt thereof, and additional therapeutically active agent(s) can be administered sequentially, either at approximately the same time or at different times, as separate compositions. When AMXT 1501, or a pharmaceutically acceptable salt thereof, and DFMO, or a pharmaceutically acceptable salt thereof, and the other therapy are administered as separate formulations or compositions, AMXT 1501, or a pharmaceutically acceptable salt thereof, and/or DFMO, or a pharmaceutically acceptable salt thereof, and the other therapy can be administered by the same route of administration or by different routes of administration. A skilled clinician can determine appropriate timing for administration of each therapy being used in combination (e.g., timing sufficient to allow an overlap of the pharmaceutical effects of the therapies).

Achieving optimal clinical benefits of a therapy (e.g., a combination therapy described herein) can be dependent on obtaining full target engagement in subject's tumors. Clinical levels of therapeutically active agents in tumor biopsies and biomarkers are convenient indicators of target engagement. Relevant biomarkers in the context of the combination therapies described herein include tumor or tumor microenvironment-associated polyamine levels (such as tumor or tumor microenvironment-associated levels of putrescine, spermidine and/or spermine), immune cell phenotype (e.g., as assessed by immunohistochemistry), immune-related gene signals (e.g., as assessed by NanoString gene expression analysis) and polyamine-related protein levels (e.g., as assessed by liquid chromatography mass spectroscopy).

Such indicators can be measured by assessing impact of treatment on the clinical level of agent and/or biomarker by comparing a pre-treatment biopsy with a biopsy taken during and/or after treatment. Part 1 of the clinical trial described in the Examples herein demonstrated tolerability, target engagement of blood polyamine uptake and adequate and sustained blood pharmacokinetic levels of AMXT 1501.

### Pharmaceutical Compositions

A therapeutically active agent described herein (e.g., AMXT 1501, or a pharmaceutically acceptable salt thereof; DFMO, or a pharmaceutically acceptable salt thereof) can be administered to a subject in pure or substantially pure form (e.g., not in admixture with another solid or liquid). Typically, for administration to a subject, an agent is formulated with one or more pharmaceutically acceptable excipients. Thus, one embodiment is a composition (e.g., pharmaceutical composition) comprising a therapeutically active agent described herein (e.g., AMXT 1501, or a pharmaceutically acceptable salt thereof; DFMO, or a pharmaceutically acceptable salt thereof) and a pharmaceutically acceptable excipient. The compositions described herein can be used in the methods described herein, e.g., to supply agent (e.g., AMXT1501 and/or DFMO, or a pharmaceutically acceptable salt thereof) for administration to a subject.

The compositions described herein may be formulated for administration by oral, parenteral (e.g., intramuscular, intraperitoneal, intravenous, intracerebroventricular, intracisternal injection or infusion, subcutaneous injection, or implant), inhalation spray, nasal, vaginal, rectal, sublingual, or topical routes of administration. In preferred embodiments, AMXT 1501, or a pharmaceutically acceptable salt thereof, or a composition comprising AMXT 1501, or a pharmaceutically acceptable salt thereof, is formulated for oral administration.

The compositions described herein may be formulated into suitable dosage forms (e.g., unit dosage forms), e.g., containing pharmaceutically acceptable excipient(s) appropriate for the intended route of administration. Accordingly, some embodiments provide a dosage form (e.g., unit dosage form) comprising a composition described herein. For example, in some embodiments, AMXT 1501, or a pharmaceutically acceptable salt thereof, is in the form of an enterically-coated, solid oral dosage form, such as an enterically-coated capsule.

Compositions described herein can be prepared by any of the methods well known in the art of pharmacy. For example, some methods include the step of bringing the active ingredient(s), e.g., AMXT 1501 and/or DFMO, or a pharmaceutically acceptable salt thereof, into association with a pharmaceutically acceptable excipient(s). In general, compositions are prepared by uniformly and intimately bringing an active ingredient(s), e.g., AMXT 1501 and/or DFMO, or a pharmaceutically acceptable salt thereof, into association with a liquid carrier or a finely divided solid carrier or both, and then, if necessary, shaping the product into the desired formulation. In the composition, the active ingredient(s), e.g., AMXT 1501 and/or DFMO, or a pharmaceutically acceptable salt thereof, is included in an amount sufficient to produce the desired effect upon the process or condition of disease. For example, in some embodiments, the concentration of an active ingredient(s), e.g., AMXT 1501 and/or DFMO, or a pharmaceutically acceptable salt thereof, in the composition is from about 0.001% to about 99%, from about 0.01% to about 98%, from about 0.1% to about 95%, from about 0.1% to about 50%, from about 0.1% to about 25%, from about 0.2% to about 20%, or from about 1% to about 10% w/w, w/v or v/v (e.g., w/w).

In one embodiment, the composition provides a therapeutically effective systemic plasma level of therapeutically active agent (e.g., AMXT 1501 and/or DFMO) for a period of at least 4, 5, 6, 7, 8, 9, 10, 11, 12, 13, 14, 15, 16, 17, 18, 19, 20, 21, 22, 23, 24, 30, 36, or 48 hours. In further embodiments, the composition provides a therapeutically effective systemic plasma level of therapeutically active agent (e.g., AMXT 1501 and/or DFMO) for at least an 8-hour period. In further embodiments, the composition provides a therapeutically effective systemic plasma level of therapeutically active agent (e.g., AMXT 1501 and/or DFMO) for at least a 12-hour period. In further embodiments, the composition provides a therapeutically effective systemic plasma level of therapeutically active agent (e.g., AMXT 1501 and/or DFMO) for at least a 14-hour period. In further embodiments, the composition provides a therapeutically effective systemic plasma level of therapeutically active agent (e.g., AMXT 1501 and/or DFMO) for at least an 18-hour period. In further embodiments, the composition provides a therapeutically effective systemic plasma level of therapeutically active agent (e.g., AMXT 1501 and/or DFMO) for at least a 24-hour period.

For example, in one embodiment, the composition provides a systemic plasma level of DFMO of greater than or about 39 μM (and, in further embodiments, of greater than or about 100 μM) for a period of at least 4, 5, 6, 7, 8, 9, 10, 11, 12, 13, 14, 15, 16, 17, 18, 19, 20, 21, 22, 23, 24, 30, 36, or 48 hours. In further embodiments, the composition provides a systemic plasma level of DFMO of greater than or about 39 μM (and, in further embodiments, of greater than or about 100 μM) for at least an 8-hour period. In further embodiments, the composition provides a systemic plasma level of DFMO of greater than or about 39 μM (and, in further embodiments, of greater than or about 100 μM) for at least a 12-hour period. In further embodiments, the composition provides a systemic plasma level of DFMO of greater than or about 39 μM (and, in further embodiments, of greater than or about 100 μM) for at least a 14-hour period. In further embodiments, the composition provides a systemic plasma level of DFMO of greater than or about 39 μM (and, in further embodiments, of greater than or about 100 PM) for at least an 18-hour period. In further embodiments, the composition provides a systemic plasma level of DFMO of greater than or about 39 μM (and, in further embodiments, of greater than or about 100 μM) for at least a 24-hour period.

In one embodiment, the composition (e.g., pharmaceutical composition) is a solid dosage form, e.g., intended for oral use. For many reasons, an oral composition, and particularly a solid oral dosage form, is advantageous and convenient for both the patient and the medical practitioner responsible for developing the therapeutic regime. An oral composition avoids the complications, cost and inconvenience of administration via IV injection or infusion which must be done by a medical professional in a hospital or outpatient setting which exposes him or her to hospital-based infections and illnesses. In particular, patients undergoing treatment for cancer may be immunocompromised individuals and particularly susceptible to hospital-based infections and illnesses. An oral formulation, such as a pill or tablet, may be taken outside of a hospital setting, increasing the potential for subject ease of use and compliance. This permits a subject to avoid infection risks concomitant with IV administration and hospital visits. In addition, oral delivery may avoid the high concentration peak and rapid clearance associated with an IV bolus dose.

Examples of oral solid dosage forms include pills, tablets, capsules, granules, and microspheres, any of which may include an enteric coating to protect the composition from acid degradation by stomach environment, or to maximize delivery to intestinal sections where absorption may be enhanced. The solid dosage form may be chewable or swallowable, or have any suitable ingestible form. In one embodiment, the solid dosage form contains little or no water, e.g., less than 0.1 weight percent water, less than 0.2 weight percent water, less than 0.3 weight percent water, less than 0.4 weight percent water, less than 0.5 weight percent water, less than 1 weight percent water, less than 1.5 weight percent water, less than 2 weight percent water or less than 5 weight percent water.

For oral administration, the compositions are preferably provided in a solid dosage form, such as the form of pills, capsules, tablets and the like, containing from about 1 milligram to about 5,000 milligrams, for example, from about 1 milligram to about 2,500 milligrams, from about 1 milligram to about 1,000 milligrams, from about 50 milligrams to about 500 milligrams or from about 50 milligrams to about 350 milligrams, particularly about: 1, 5, 10, 15, 20, 25, 50, 75, 100, 150, 200, 250, 300, 400, 500, 600, 750, 800, 900, or 1,000 milligrams, of active ingredient(s) (e.g., AMXT 1501 and/or DFMO, or a pharmaceutically acceptable salt thereof).

In one embodiment, a solid dosage form contains about: 10 mg, or 20 mg, or 30 mg, or 40 mg, or 50 mg, or 60 mg, or 70 mg, or 80 mg, or 90 mg, or 100 mg, or 110 mg, or 120 mg, or 130 mg, or 140 mg, or 150 mg, or 200 mg, or 250 mg, or 300 mg, or 350 mg, or 400 mg, or 450 mg, or 500 mg, of AMXT 1501, or a pharmaceutically acceptable salt thereof, based on AMXT 1501 free base. The amount of AMXT 1501, or a pharmaceutically acceptable salt thereof, present in a solid dosage form may also be characterized in terms of a range of possible amounts, where lower and upper limits of the range are selected from the amounts just described, e.g., from about 10 mg to about 500 mg, or numbers in between, e.g., from about 50 to about 350 mg, based on AMXT 1501 free base. A tablet or pill will typically have a total weight of at least 50 mg.

In one embodiment, the solid dosage form provides a therapeutically effective systemic plasma level of therapeutically active agent (e.g., AMXT 1501 and/or DFMO) for a period of at least 4, 5, 6, 7, 8, 9, 10, 11, 12, 13, 14, 15, 16, 17, 18, 19, 20, 21, 22, 23, 24, 30, 36, or 48 hours. In further embodiments, the solid dosage form provides a therapeutically effective systemic plasma level of therapeutically active agent (e.g., AMXT 1501 and/or DFMO) for at least an 8-hour period. In further embodiments, the solid dosage form provides a therapeutically effective systemic plasma level of therapeutically active agent (e.g., AMXT 1501 and/or DFMO) for at least a 12-hour period. In further embodiments, the solid dosage form provides a therapeutically effective systemic plasma level of therapeutically active agent (e.g., AMXT 1501 and/or DFMO) for at least a 14-hour period. In further embodiments, the solid dosage form provides a therapeutically effective systemic plasma level of therapeutically active agent (e.g., AMXT 1501 and/or DFMO) for at least an 18-hour period. In further embodiments, the solid dosage form provides a therapeutically effective systemic plasma level of therapeutically active agent (e.g., AMXT 1501 and/or DFMO) for at least a 24-hour period.

For example, in one embodiment, the solid dosage form provides a systemic plasma level of AMXT 1501 of at least or about 50 nM (and, in further embodiments of at least or about 100 nM) for a period of at least 4, 5, 6, 7, 8, 9, 10, 11, 12, 13, 14, 15, 16, 17, 18, 19, 20, 21, 22, 23, 24, 30, 36, or 48 hours. In further embodiments, the solid dosage form provides a systemic plasma level of AMXT 1501 of at least or about 50 nM (and, in further embodiments of at least or about 100 nM) for at least an 8-hour period. In further embodiments, the solid dosage form provides a systemic plasma level of AMXT 1501 of at least or about 50 nM (and, in further embodiments of at least or about 100 nM) for at least a 12-hour period. In further embodiments, the solid dosage form provides a systemic plasma level of AMXT 1501 of at least or about 50 nM (and, in further embodiments of at least or about 100 nM) for at least a 14-hour period. In further embodiments, the solid dosage form provides a systemic plasma level of AMXT 1501 of at least or about 50 nM (and, in further embodiments of at least or about 100 nM) for at least an 18-hour period. In further embodiments, the solid dosage form provides a systemic plasma level of AMXT 1501 of at least or about 50 nM (and, in further embodiments of at least or about 100 nM) for at least a 24-hour period.

Solid dosage forms may be prepared according to any method known to the art for the manufacture of such. Such compositions may contain one or more inert components, where exemplary inert components may be selected from the group of sweetening agents, flavoring agents, coloring agents and preserving agents.

Excipients for solid dosage forms are well known in the art, and are selected to provide various benefits including, for example, ease of administration to the subject, improved dosing compliance, consistency and control of drug bioavailability, assistance with enhanced bioavailability, improved API stability including protection from degradation, and to contribute to the ease of production of a robust and reproducible physical product. Excipients are commonly subdivided into various functional classifications, depending on the role that they are intended to play in the formulation. For solid dosage forms, common excipient roles and exemplary materials that fulfill that role are diluents, e.g., lactose and microcrystalline cellulose, disintegrants, e.g., sodium starch glycolate and croscarmellose sodium, binders, e.g., PVP and HPMC, lubricants, e.g., magnesium stearate, and glidants, e.g., colloidal SiO₂. Tablets and capsules often contain a diluent, filler and/or bulking agent, e.g., lactose. Excipients used to formulate the compositions described herein should typically avoid those containing reducing sugar components in order to prevent formation of Schiff-base addition products as degradants.

Excipients may be, for example, inert diluents, such as calcium carbonate, sodium carbonate, and lactose; granulating and disintegrating agents, such as corn starch and alginic acid; binding agents, such as starch, gelatin or acacia; and lubricating agents, such as magnesium stearate, stearic acid and talc.

Tablets may be uncoated or they may be coated, e.g., with an enteric coating, by known techniques, in order to delay disintegration and absorption in the gastrointestinal tract and thereby provide a sustained action over a longer period. Compositions for oral use may also be formed as hard gelatin capsules wherein the active ingredient(s), e.g., AMXT 1501 and/or DFMO, or a pharmaceutically acceptable salt thereof, is mixed with an inert solid diluent, for example, calcium carbonate, or kaolin, or as soft gelatin capsules wherein the active ingredient(s), e.g., AMXT 1501 and/or DFMO, or a pharmaceutically acceptable salt thereof, is mixed with water or an oil medium, for example, peanut oil, liquid paraffin, or olive oil.

Compositions described herein can also be in the form of aqueous suspensions. Oily suspensions may be formulated by suspending the active ingredient in a suitable oil. Oil-in-water emulsions may also be employed. Dispersible powders and granules suitable for preparation of an aqueous suspension by the addition of water may also be used to provide an active ingredient(s), e.g., AMXT 1501 and/or DFMO, or a pharmaceutically acceptable salt thereof, in admixture with a dispersing or wetting agent, suspending agent and one or more preservatives.

Pharmaceutical compositions may be in the form of a sterile injectable aqueous or oleaginous suspension or a suppository for rectal administration. For topical use, creams, ointments, jellies, solutions or suspensions, etc., containing a therapeutic agent(s) described herein, e.g., AMXT 1501 and/or DFMO, or a pharmaceutically acceptable salt thereof, may be employed. Compositions may also be formulated for administration by inhalation or a transdermal patch by methods known in the art.

Compositions (e.g., compositions comprising DFMO, or a pharmaceutically acceptable salt thereof) can also be administered subcutaneously, intraperitoneally or intravenously. Such compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation. Compositions described herein for intravenous, subcutaneous, or intraperitoneal injection may contain an isotonic vehicle such as sodium chloride injection, Ringer's injection, dextrose injection, dextrose and sodium chloride injection, lactated Ringer's injection, water such as water for injection, or other vehicles known in the art.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs. For example, topical application for the lower intestinal tract can be effected in a rectal suppository formulation or in a suitable enema formulation. Topically-transdermal patches can also be used. For other topical applications, the compositions can be formulated in a suitable ointment containing the active component suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water. In other embodiments, suitable carriers include, but are not limited to, mineral oil, sorbitan monostearate, polysorbate 60, cetyl esters wax, cetearyl alcohol, 2-octyldodecanol, benzyl alcohol and water and penetration enhancers. For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

In some embodiments, a composition may further comprise one or more additional therapeutically active agents, e.g., which are beneficially applied in the treatment of a disease, disorder or condition, such as cancer, experienced or potentially experienced by a subject receiving a therapeutic agent(s) described herein, e.g., AMXT 1501 and/or DFMO, or a pharmaceutically acceptable salt thereof.

As mentioned above, a composition may be formulated as a tablet, capsule or the like. For example, in one embodiment, a composition comprises from about 0.1% to about 50% of AMXT 1501, or a pharmaceutically acceptable salt thereof (e.g., AMXT 1501 dicaprate); from about 0.1% to about 99.9% of a filler; from about 0% to about 10% of a disintegrant; from about 0% to about 5% of a lubricant; and from about 0% to about 5% of a glidant. For example, in one embodiment, a composition comprises from about 0.1% to about 50% of AMXT 1501, or a pharmaceutically acceptable salt thereof (e.g., AMXT 1501 dicaprate); from about 0.1% to about 99.9% of a filler; from about 0% to about 10% of a disintegrant; from about 0% to about 5% of a lubricant; and from about 0% to about 5% of a glidant. Optionally, a composition (e.g., in unit dosage form) comprises from about 10 mg to about 300 mg of AMXT 1501, or a pharmaceutically acceptable salt thereof (e.g., AMXT 1501 dicaprate), making up from about 2% to about 50% of the tablet content or capsule fill content, for example; from about 0% to about 10% of a disintegrant; from about 0% to about 5% of a lubricant; from about 0% to about 5% of a glidant; and from about 30% to about 98% of a filler. In another embodiment, a composition comprises AMXT 1501, or a pharmaceutically acceptable salt thereof (e.g., AMXT 1501 dicaprate), from about 0.1% to about 10% of a binder, from about 0% to about 5% of a surfactant, from about 0% to about 10% of an intergranular disintegrant, and from about 0% to about 10% of an extragranular disintegrant.

Examples of binders, fillers, surfactants, disintegrants, lubricants, intergranular disintegrants, extragranular disintegrants and glidants are known in the art, and examples are disclosed herein, and include, a binder selected from copolyvidone, hydroxypropylcellulose, hydroxypropylmethylcellulose, and povidone; a filler selected from a sugar, a starch, a cellulose, and a poloxamer; a surfactant selected from polyoxyethylene sorbitan monooleate, a poloxamer, and sodium lauryl sulfate; an intergranular disintegrant selected from croscarmellose sodium, sodium starch glyconate, and crospovidone. Other examples include a disintegrant selected from povidone and crospovidone; a lubricant which is magnesium stearate; and a glidant which is silicon dioxide.

For example, in one embodiment, provided is an oral pharmaceutical composition, preferably a solid dosage form, comprising AMXT 1501, or a pharmaceutically acceptable salt thereof (e.g., AMXT 1501 dicaprate) and at least one oral pharmaceutically acceptable excipient, which yields a therapeutically effective systemic plasma AMXT 1501 level for at least a 12-hour period when orally administered to a subject. The composition may be further characterized by one or more of the following: the composition yields a therapeutically effective systemic plasma concentration of AMXT 1501 level for at least a 24-hour period when orally administered to a subject; the plasma level of AMXT 1501 is at least 75% of the peak plasma concentration for 4 or more hours; the composition has an oral bioavailability of at least 1%, or at least 3%, or at least 4%; the composition yields a therapeutically effective plasma level of AMXT 1501 for at least a 24-hour period in the subject with once-daily dosing; the composition has a half-life of at least 12 hours or at least 18 hours; the composition does not have substantially dose-limiting side effects, e.g., gastrointestinal side effects such as nausea, vomiting, diarrhea, abdominal pain, oral mucositis, oral ulceration, pharyngitis, stomatitis, or gastrointestinal ulceration; the composition comprises about 25 mg to about 350 mg of AMXT 1501 in salt form; and/or the composition is in the form of a tablet or capsule.

Kits are also provided in accordance with the present disclosure. In some embodiments, a kit comprises an oral dosage form of AMXT 1501, or a pharmaceutically acceptable salt thereof, and DFMO, or a pharmaceutically acceptable salt thereof (e.g., a composition comprising DFMO, such as Ornidyl). In some embodiments, the kit further comprises a diluent for the DFMO, or a pharmaceutically acceptable salt thereof, such as water (e.g., water for injection (WFI)), dextrose or saline. In some embodiments, the kit further comprises an ambulatory infusion system, such as an ambulatory infusion system described herein, for dispensing the DFMO, or a pharmaceutically acceptable salt thereof, or a composition comprising DFMO, or a pharmaceutically acceptable salt thereof, formulated for intravenous administration. Written instructions for administering AMXT 1501, or a pharmaceutically acceptable salt thereof, and DFMO, or a pharmaceutically acceptable salt thereof, to a subject to treat a disease, disorder or condition described herein, such as cancer, can also be provided in the kit.

Preferably, the compositions provided herein do not have substantially dose-limiting side effects, e.g., gastrointestinal side effects such as nausea, vomiting, diarrhea, abdominal pain, oral mucositis, oral ulceration, pharyngitis, stomatitis, and gastrointestinal ulceration.

### EXAMPLES

The Examples provided below further illustrate and exemplify the subject matter described herein. It is to be understood that the following Examples are not intended to limit the scope of the subject matter described herein in any way. In the following Examples, molecules with a single chiral center, unless otherwise noted, exist as a racemic mixture. Those molecules with two or more chiral centers, unless otherwise noted, can exist as a racemic mixture of diastereomers. Single enantiomers/diastereomers may be obtained by methods known to those skilled in the art. The starting materials and various reactants utilized or referenced in the Examples may be obtained from commercial sources, or are readily prepared from commercially available organic compounds, using methods well known to those skilled in the art.

### Example 1: Oral AMXT 1501 Dicaprate in Combination with Oral DFMO

Aminex Therapeutics is developing oral AMXT 1501 dicaprate in combination with oral DFMO for treatment of cancer patients (clinicaltrials.gov NCT03536728). The objective of this clinical trial is to determine the safety and tolerability of oral AMXT 1501 dicaprate in combination with DFMO in patients with advanced solid tumors. Interim data for the pharmacokinetic (PK) behavior of both agents in human subjects has been generated. Both AMXT 1501 dicaprate and DFMO have high water solubility. Due to low bioavailability of AMXT 1501 dicaprate, this agent appears to belong to Biopharmaceutics Classification System (BCS) Class 3.

Clinical Trial Outline. Aminex's clinical trial evaluating the combination of AMXT 1501 dicaprate and DFMO consists of two parts. All solid tumor types are eligible for the trial. Part 1 of the clinical trial evaluated the safety of escalating doses of AMXT 1501 dicaprate, given alone, for two weeks to patients with cancer, then given in combination with a fixed low dose of DFMO for an additional two weeks. A standard 3+3 AMXT 1501 dicaprate dose escalation design was utilized. In Part 2 of the clinical trial, the AMXT 1501 dicaprate dose was fixed at 1,200 mg (calculated as the free base content of AMXT 1501 dicaprate), and the DFMO dose was escalated, following a 3+3 dose escalation protocol design, to determine the maximum tolerated dose (MTD) and recommended Phase 2 dose (RP2D) for the combination. An expansion cohort was included at the RP2D level to confirm safety and tolerability of the combination. In Part 1 of this clinical trial, AMXT 1501 dicaprate was delivered to fasted patients orally, once daily in enteric-coated capsules. DFMO was delivered orally twice daily in gel capsules. In Part 2, patients were treated by giving both agents twice daily (BID dosing schedule). Changing from once daily to twice daily oral delivery of AMXT 1501 dicaprate was driven by gastrointestinal (GI) adverse effects observed in several Part 1 patients treated with high once daily dose levels of AMXT 1501 dicaprate. Upon changing to twice daily delivery (BID), these GI adverse effects were greatly reduced, and PK evaluation of plasma levels of AMXT 1501 highlighted that sustained and higher than effective dose levels of AMXT 1501 were still obtained.

Fifty-two human cancer patients have been orally treated with AMXT 1501 dicaprate in enterically-coated capsules (e.g., described in Example 6 herein), with or without oral DFMO. Plasma AMXT 1501 and DFMO concentration versus nominal time data are available from Cohorts 1 through 5 of Part 1 of the clinical trial (corresponding to dose levels of 80, 160, 400, 1,200, and 1,800 mg AMXT 1501 alone or in combination with DFMO 500 mg total daily, split BID). Twenty-one patients have been dosed in Part 1. Based on the interim pharmacokinetic data from Part 1, mean AMXT 1501 exposure following single or repeat PO dosing increased in a dose-dependent manner at dose levels of 160 mg AMXT 1501 and higher.

Interim AMXT 1501 and DFMO Clinical Pharmacokinetic (PK) Data. Plasma levels of AMXT 1501 and DFMO were determined using GLP validated LC/MS² bioanalytical methods. Plasma samples were analyzed for AMXT 1501 concentration via a LC/MS² procedure (MI-0028-DB-AB; Agilux Laboratories, Worcester, Massachusetts), and the resulting concentration versus time data were used to estimate individual PK parameters using noncompartmental analysis (NCA).

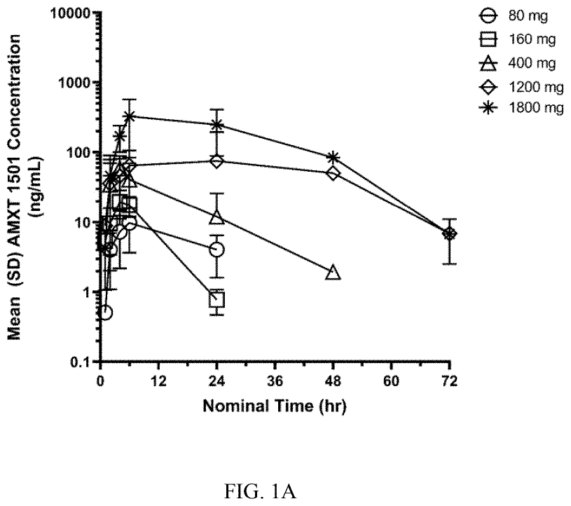

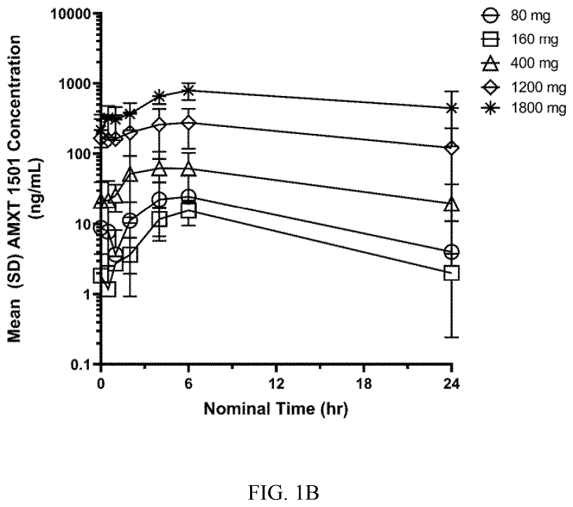

Blood samples from patients were collected over the first three days after a single dose of AMXT 1501 dicaprate on Day 1 of the study, and over a 24-hour period on Day 14 of the study after repeat dosing of AMXT 1501 dicaprate according to the study design. FIGS. 1A and 1B show the resulting AMXT 1501 mean plasma levels from the study.

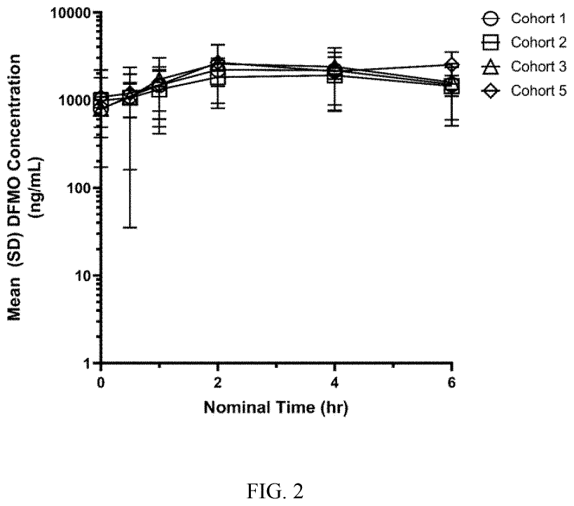

Twenty patients were treated with the 500 mg/day DFMO (split twice daily) from Day 15-28 of the study. DFMO plasma levels were measured at various times post-dosing on Day 28 of the study, and the resultant DFMO plasma concentrations were determined using a validated LC/MS² bioanalytical assay. Resulting DFMO plasma levels versus time for all 20 patients post-dosing are shown in FIG. 2. All these patients were treated with same DFMO dose and showed an AUC (n=14) average of 11,417 hr*ng/mL, SD 4,870, for a percent CV of 43%.

At the highest AMXT 1501 dose provided to patients (Part 1, Cohort 5: 1,800 mg AMXT 1501), an average Cₘₐₓ of 794 ng/mL (SD 209, for a percent CV of 26%) was observed for plasma AMXT 1501 concentration after repeat dosing (Day 14; n=4 patients). Plasma levels of AMXT 1501 were sustained over the 24-hour dosing time increment. An average AUC value of 14,225 hr*ng/mL (SD 5,017 hr*ng/mL, for a percent CV of 35%) was observed for these four patients in Part 1, Cohort 5.

It was found that splitting the once daily dose frequency (QD), to a twice per day dose schedule (BID) substantially minimized the occurrence of sometimes moderate GI-associated adverse effects (e.g., nausea, diarrhea and, less frequently, vomiting) at similar dose levels. As determined by the plasma versus time pharmacokinetic graphs, this dose splitting had minimal effects on patient plasma drug exposure. In fact, delivery of AMXT 1501 dicaprate via oral delivery to human cancer patients has shown sustained, durable and more than adequate drug agent exposure over the daily dosing frequency. Furthermore, pharmacodynamic measurements have demonstrated robust and sustained polyamine transporter engagement by AMXT 1501 in human patients.

Mean AMXT 1501 exposure following single or repeat PO dosing increased in a dose-dependent manner at dose levels of 160 mg and higher. Inter-subject variability in AMXT 1501 exposure as well as the level of accumulation with repeat dosing was high. Part of this variability could have been related to differences in patient body weights, as both AMXT 1501 and DFMO were administered as fixed doses. To date, there has been no obvious effect of co-administration of DFMO on the pharmacokinetics (PK) of AMXT 1501. The Cohort 5 AMXT 1501 dose level of 1,800 mg/day was selected as the starting AMXT 1501 Part 2 dose based on reaching the Part 1 stopping criterion of plasma AMXT 1501 Cₘₐₓ of 1000 ng/mL. In Part 2, increasing doses of DFMO were evaluated with a fixed AMXT 1501 dose level based on Part 1 AMXT 1501 tolerability and PK results.

Multiple positive murine efficacy studies have dosed AMXT 1501 dicaprate at 2.5 mg/kg by daily subcutaneous injections. PK assessment of this same dose in 4T1 breast cancer syngeneic tumor-bearing mice showed AMXT 1501 plasma Cₘₐₓ values reached 978 ng/mL, with AUC values of 8,270 hr*ng/mL. Importantly, in this mouse study (described in Example 3), tumor retention of AMXT 1501 was 18 times higher than plasma, with AUC values of 152,000 hr*ng/mL. Tumor AMXT 1501 levels versus plasma AMXT 1501 levels were 894 times higher at the 72-hour time point, further highlighting the high tumor versus plasma specificity of AMXT 1501 in the 4T1 model. Although not wishing to be bound by any particular theory, the difference between the plasma AUC value and the tumor AUC value in mice suggests that plasma AMXT 1501 levels observed in the clinical trial are an underestimation of tumor target engagement. Patients in Cohort 5 had AUC values 1.72 times higher than plasma levels inferred by these data in multiple efficacious mouse studies. These data support the conclusion that sufficient and comparable AMXT 1501 plasma levels have been observed in human cancer patients versus animals at dose levels that provided robust efficacy results in multiple mouse tumor efficacy models.

For PK characterization, it is generally assumed that the Cₘa parameter, the maximal plasma level of drug, tracks toxicological endpoints, while the parameter of AUC, measuring sustained plasma drug concentrations, tracks efficacy endpoints. PK data obtained in patients in Part 1 of the trial suggest robust AMXT 1501 plasma coverage, as reflected by the AUC values. Furthermore, sustained, 24-hour plasma levels of AMXT 1501 were observed over the dosing time increment. Given the modest Grades 1 and 2 adverse effects observed in these patients, mainly gastrointestinal in nature (nausea and diarrhea), it is presumed that plasma Cₘa values below 1,000 ng/mL AMXT 1501 are well-tolerated. Importantly, in vitro EC₅₀ values for AMXT 1501 against cancer cells are 51 nM, which is equivalent to a 28 ng/mL plasma concentration of AMXT 1501. See Burns, M. R., et al., J Med Chem 52, 1983-1993 (2009), the entire content of which is incorporated herein by reference.

A statistical summary of the PK data from Part 1 of the clinical trial appears in Table 1.1. The analyte measured in all cases was AMXT 1501, and the regimen was QD.

TABLE 1.1

| | | Daily | | Nominal | | | | | | | | | |
|---|---|---|---|---|---|---|---|---|---|---|---|---|---|
| | | Dose | | Dosing | | | | | | | | | CV |
| Part | Cohort | (mg) | Cycle | Day | Parameter | Units | Z | Mean | SD | Min | Median | Max | % |
| 1 | 1 | 80 | 1 | 1 | Tₘₐₓ | hr | 5 | 4.8 | 1.1 | 4 | 4 | 6 | 22.8 |
| | | | | | Cₘₐₓ | ng/ml | 5 | 11.7 | 5.22 | 6.79 | 10.6 | 19.8 | 44.6 |
| | | | | | AUC_{0-6 hr} | hr*ng/ml | 5 | 29.6 | 10.9 | 15.9 | 29.9 | 45.3 | 36.9 |
| | | | | | Tₗₐₛₜ | hr | 5 | 13.2 | 9.86 | 6 | 6 | 24 | 74.7 |
| | | | | | AUC₀₋ₜ | hr*ng/ml | 5 | 92 | 94.6 | 29.9 | 45.3 | 254 | 103 |
| 1 | 1 | 80 | 1 | 14 | Tₘₐₓ | hr | 3 | 4.67 | 1.15 | 4 | 4 | 6 | 24.7 |
| | | | | | Cₘₐₓ | ng/ml | 3 | 26.2 | 23.2 | 11.8 | 13.8 | 53 | 88.7 |
| | | | | | AUC_{0-6 hr} | hr*ng/ml | 3 | 91 | 78.5 | 40.2 | 51.3 | 181 | 86.3 |
| | | | | | Tₗₐₛₜ | hr | 3 | 12 | 10.4 | 6 | 6 | 24 | 86.6 |
| | | | | | AUC₀₋ₜ | hr*ng/ml | 3 | 262 | 375 | 40.2 | 51.3 | 694 | 143 |
| 1 | 1 | 80 | 1 | 28 | Tₘₐₓ | hr | 3 | 11.3 | 11 | 4 | 6 | 24 | 97.2 |
| | | | | | Cₘₐₓ | ng/ml | 3 | 16.4 | 16.2 | 0.945 | 15.1 | 33.2 | 98.5 |
| | | | | | AUC_{0-6 hr} | hr*ng/ml | 3 | 60.5 | 58.7 | 0.709 | 62.9 | 118 | 96.9 |
| | | | | | Tₗₐₛₜ | hr | 3 | 18 | 10.4 | 6 | 24 | 24 | 57.7 |
| | | | | | AUC₀₋ₜ | hr*ng/ml | 3 | 169 | 229 | 11.3 | 62.9 | 431 | 136 |
| 1 | 2 | 160 | 1 | 1 | Tₘₐₓ | hr | 4 | 4 | 0 | 4 | 4 | 4 | 0 |
| | | | | | Cₘₐₓ | ng/ml | 4 | 19.5 | 4.96 | 15.6 | 17.8 | 26.7 | 25.5 |
| | | | | | AUC_{0-6 hr} | hr*ng/ml | 4 | 74.8 | 25.7 | 53.3 | 67.1 | 112 | 34.3 |
| | | | | | Tₗₐₛₜ | hr | 4 | 15 | 10.4 | 6 | 15 | 24 | 69.3 |
| | | | | | AUC₀₋ₜ | hr*ng/ml | 4 | 169 | 135 | 63.5 | 130 | 352 | 79.9 |
| 1 | 2 | 160 | 1 | 14 | Tₘₐₓ | hr | 4 | 5 | 1.15 | 4 | 5 | 6 | 23.1 |
| | | | | | Cₘₐₓ | ng/ml | 4 | 14.1 | 6.73 | 4.81 | 16.2 | 19.1 | 47.8 |
| | | | | | AUC_{0-6 hr} | hr*ng/ml | 4 | 43.4 | 18.6 | 17 | 49.8 | 5 | 42.8 |
| | | | | | Tₗₐₛₜ | hr | 4 | 15 | 10.4 | 6 | 15 | 24 | 69.3 |
| | | | | | AUC₀₋ₜ | hr*ng/ml | 4 | 105 | 102 | 43.8 | 59.7 | 257 | 96.8 |
| 1 | 2 | 160 | 1 | 28 | Tₘₐₓ | hr | 4 | 4.5 | 1 | 4 | 4 | 6 | 22.2 |
| | | | | | Cₘₐₓ | ng/ml | 4 | 23.7 | 6.79 | 16.5 | 23.2 | 31.8 | 28.7 |
| | | | | | AUC_{0-6 hr} | hr*ng/ml | 4 | 88.6 | 32.5 | 54 | 84.8 | 131 | 36.7 |
| | | | | | Tₗₐₛₜ | hr | 4 | 10.5 | 9 | 6 | 6 | 24 | 85.7 |
| | | | | | AUC₀₋ₜ | hr*ng/ml | 4 | 150 | 119 | 54 | 112 | 323 | 79.2 |
| 1 | 3 | 400 | | 1 | Tₘₐₓ | hr | 4 | 5 | 1.15 | 4 | 5 | 6 | 23.1 |
| | | | | | Cₘₐₓ | ng/ml | 4 | 47 | 35.7 | 1.73 | 52.5 | 81.4 | 76 |
| | | | | | AUC_{0-6 hr} | hr*ng/ml | 4 | 183 | 154 | 5.19 | 207 | 314 | 84 |
| | | | | | Tₗₐₛₜ | hr | 4 | 25.5 | 17.2 | 6 | 24 | 48 | 67.6 |
| | | | | | AUC₀₋ₜ | hr*ng/ml | 4 | 714 | 477 | 5.19 | 906 | 1040 | 66.8 |
| 1 | 3 | 400 | 1 | 14 | Tₘₐₓ | hr | 4 | 5 | 1.15 | 4 | 5 | 6 | 23.1 |
| | | | | | Cₘₐₓ | ng/ml | 4 | 66.4 | 43.9 | 6.65 | 73.5 | 112 | 66.1 |
| | | | | | AUC_{0-6 hr} | hr*ng/ml | 4 | 289 | 212 | 16.5 | 304 | 532 | 73.5 |
| | | | | | Tₗₐₛₜ | hr | 4 | 19.5 | 9 | 6 | 24 | 24 | 46.2 |
| | | | | | AUC₀₋ₜ | hr*ng/ml | 4 | 957 | 692 | 16.5 | 1100 | 1610 | 72.4 |
| 1 | 3 | 400 | 1 | 28 | Tₘₐₓ | hr | 3 | 11.3 | 11 | 4 | 6 | 24 | 97.2 |
| | | | | | Cₘₐₓ | ng/ml | 3 | 55.8 | 65.2 | 16.1 | 20.3 | 131 | 117 |
| | | | | | AUC_{0-6 hr} | hr*ng/ml | 3 | 234 | 275 | 57.5 | 93.8 | 550 | 117 |
| | | | | | Tₗₐₛₜ | hr | 3 | 24 | 0 | 24 | 24 | 24 | 0 |
| | | | | | AUC₀₋ₜ | hr*ng/ml | 3 | 791 | 797 | 246 | 422 | 1710 | 101 |
| 1 | 4 | 1200 | 1 | 1 | Tₘₐₓ | hr | 3 | 11.3 | 11 | 4 | 6 | 24 | 97.2 |
| | | | | | Cₘₐₓ | ng/ml | 3 | 104 | 96.9 | 27.8 | 71.2 | 213 | 93.1 |
| | | | | | AUC_{0-6 hr} | hr*ng/ml | 3 | 197 | 134 | 47.1 | 238 | 306 | 68.1 |
| | | | | | Tₗₐₛₜ | hr | 3 | 40 | 27.7 | 24 | 24 | 72 | 69.3 |
| | | | | | AUC₀₋ₜ | hr*ng/ml | 3 | 2730 | 3690 | 380 | 831 | 6980 | 135 |
| 1 | 4 | 1200 | 1 | 14 | Tₘₐₓ | hr | 3 | 6 | 0 | 6 | 6 | 6 | 0 |
| | | | | | Cₘₐₓ | ng/ml | 3 | 276 | 158 | 148 | 227 | 453 | 57.4 |
| | | | | | AUC_{0-6 hr} | hr*ng/ml | 3 | 1130 | 887 | 561 | 681 | 2150 | 78.4 |
| | | | | | Tₗₐₛₜ | hr | 3 | 24 | 0 | 24 | 24 | 24 | 0 |
| | | | | | AUC₀₋ₜ | hr*ng/ml | 3 | 4700 | 3290 | 2190 | 3490 | 8420 | 69.9 |
| 1 | 5 | 1800 | 1 | 1 | Tₘₐₓ | hr | 4 | 10.5 | 9 | 6 | 6 | 24 | 85.7 |
| | | | | | Cₘₐₓ | ng/ml | 4 | 367 | 219 | 95.3 | 374 | 626 | 59.7 |
| | | | | | AUC_{0-6 hr} | hr*ng/ml | 4 | 750 | 442 | 327 | 662 | 1350 | 58.9 |
| | | | | | Tₗₐₛₜ | hr | 4 | 54 | 23 | 24 | 60 | 72 | 42.6 |
| | | | | | AUC₀₋ₜ | hr*ng/ml | 4 | 10200 | 6090 | 1280 | 12700 | 14100 | 59.7 |
| 1 | 5 | 1800 | 1 | 14 | Tₘₐₓ | hr | 4 | 5.5 | 1 | 4 | 6 | 6 | 18.2 |
| | | | | | Cₘₐₓ | ng/ml | 4 | 794 | 209 | 606 | 766 | 1040 | 26.3 |
| | | | | | AUC_{0-6 hr} | hr*ng/ml | 4 | 3110 | 801 | 2390 | 3020 | 4020 | 25.8 |
| | | | | | Tₗₐₛₜ | hr | 4 | 24 | 0 | 24 | 24 | 24 | 0 |
| | | | | | AUC₀₋ₜ | hr*ng/ml | 4 | 14200 | 4990 | 10100 | 12800 | 21200 | 35.1 |
| 1 | 5 | 1800 | 1 | 28 | Tₘₐₓ | hr | 3 | 4.67 | 1.15 | 4 | 4 | 6 | 24.7 |
| | | | | | Cₘₐₓ | ng/ml | 3 | 401 | 249 | 202 | 321 | 681 | 62.1 |
| | | | | | AUC_{0-6 hr} | hr*ng/ml | 3 | 1860 | 1020 | 1120 | 1430 | 3020 | 55 |
| | | | | | Tₗₐₛₜ | hr | 3 | 24 | 0 | 24 | 24 | 24 | 0 |
| | | | | | AUC₀₋ₜ | hr*ng/ml | 3 | 7370 | 4260 | 3350 | 6920 | 11800 | 57.8 |
| 1 | 5 | 1800 | 2 | 14 | Tₘₐₓ | hr | 1 | 6 | | 6 | 6 | 6 | |
| | | | | | Cₘₐₓ | ng/ml | 1 | 1950 | | 1950 | 1950 | 1950 | |
| | | | | | AUC_{0-6 hr} | hr*ng/ml | 1 | 7240 | | 7240 | 7240 | 7240 | |
| | | | | | Tₗₐₛₜ | hr | 1 | 24 | | 24 | 24 | 24 | |
| | | | | | AUC₀₋ₜ | hr*ng/ml | 1 | 33700 | | 33700 | 33700 | 33700 | |

Clinical Drug Ratios. Table 1.2 depicts the doses of AMXT 1501 and DFMO used tumor target susceptibility, drug tumor distribution and retention and genetic background of the patient and the tumor. Given its goal of demonstration of tolerability and maximally-achievable dose of the two drug agents, Aminex's initial clinical trial dosed patients with a fixed, unit dose level of each agent. It is expected drug dosing for future clinical trials will be performed on the more precise mg/m² unit basis. Dosing based on m², also known as Body Surface Area (BSA), tends to be more precise, given it adjusts dose to patient BSA, and indirectly to patient body weight.

TABLE 1.2
AMXT 1501 - DFMO Drug Ratios in Aminex NCT03536728
First-in-Human Clinical Trial.

| | | DFMO |
|---|---|---|
| | | dose (mg), |
| | AMXT 1501 | all split |
| Cohort | dose (mg) | BID |
| Part 1 - | 1800 (QD) | 500 |
| Cohort 5 | | |
| Part 2 - | 1800 (QD) | 1000 |
| Cohort 1 | | |
| Part 2 - | 800 (BID) | 1000 |
| Cohort 2 | | |
| Part 2 - | 1200 (BID) | 1000 |
| Cohort 3 | | |
| Part 2 - | 1200 (BID) | 2000 |
| Cohort 4 | | |
| Part 2 - | 1200 (BID) | 4000 |
| Cohort 5 | | |

Three of four Part 2, Cohort 4 patients in Aminex's AMXT 1501 plus DFMO Phase 1 trial (AMXT1501-101A) experienced dose-limiting toxicities (DLTs) at an oral dose level of 1,200 mg AMXT 1501 dicaprate split BID, and 2,000 mg of DFMO, also split BID. These DLTs involved vomiting, diarrhea and nausea, and inferred a potential dual drug targeting of the gastrointestinal (GI) tract in these patients. One patient experienced severe dehydration, requiring hospitalization. A similar observation was made in dogs in Aminex's 28-day toxicological testing. The experience of Part 2, Cohort 4 patients limits orally tolerated dose amounts of AMXT 1501 and DFMO, used in combination. The clinical observations and the data obtained in dog studies, together, support the hypothesis that combined oral drug dosing levels and DFMO targeting of the GI tract are causing the intolerance.

Oral delivery of 1,200 mg AMXT 1501 dicaprate alone, either once daily, or split BID, is well tolerated clinically. In cohorts before Part 2, Cohort 4, 14 adults were treated at 1,200 mg or higher of AMXT 1501 daily, either once daily or BID, with no DLTs. A total of 18 patients were treated at 800 mg AMXT 1501 or higher daily with no DLTs. These patients included a couple of adult patients where Cₘₐₓ values above 1,000 ng/mL AMXT 1501 plasma levels were observed, again with no DLTs observed. Many of these patients were treated with multiple 28-day cycles of combined AMXT 1501 dicaprate plus DFMO therapy; several patients received over 8 courses uninterrupted. These data, too, support the hypothesis that tolerability issues in Part 2, Cohort 4 patients are due to dual drug targeting of the polyamine pathway in the GI tract of these patients.

It is well-known that cell turnover in the GI mucosal layer is extremely fast (2-3 days) and polyamine metabolism of the gut mucosal is critically important for normal, healthy cell turnover. It is also well-established that polyamines regulate the expression of c-myc, which plays a critical role in stimulation of normal intestinal epithelial cell proliferation.

In vitro AMXT 1501 ED₅₀ average against four tumor cell lines was 51 nM. Therefore, an ED₉₀ level is estimated at 100 nM, or 57 ng/mL. All adult patients treated at 1,200 mg or 800 mg per day, split BID, saw sustained plasma PK levels of AMXT 1501 above this predicted efficacious concentration value. Pharmacodynamic analysis of treated adults showed sustained AMXT 1501 target engagement using an ex vivo FACS-based assay of polyamine uptake inhibition. Animal data highlighting the high AMXT 1501 tumor levels versus plasma levels further support that adequate tumor AMXT 1501 drug levels have been achieved.

For DFMO, an in vitro Kᵢ value of 39 μM against ornithine decarboxylase (ODC) isolated enzyme has been reported. From this Kᵢ value, an ED₅₀ value of 39 μM (7,098 ng/mL) can be estimated. An efficacious plasma level for DFMO can be suggested to be 100 μM (18,200 ng/mL), which corresponds to the estimated ED₉₀ value. An average clinical Cₘₐₓ value for DFMO after 7-day repeat dosing of 1000 mg, split BID, of 4,440 ng/mL (n=6; SD+/−1,990, % CV of 44.7) was observed in Cohort 3 patients. DFMO showed PK variability and levels well below predicted efficacious plasma levels. These data strongly suggest that DFMO's target, ODC, at the Cₘₐₓ values observed for these Cohort 3 patients, let alone oral PK trough Cₘᵢₙ values, was not fully engaged. Given the short half-life and quick turnover of ODC, sustained DFMO levels, over at least its ED₅₀ value, and hopefully above its ED₉₀ value, have been strongly recommended in the literature.

The high DFMO levels used against tumor cells in tissue culture experiments is a concern. Generally, 2-5 mM levels are used (50× higher than the Kᵢ value given above), implying that high concentrations are required to ensure complete engagement of intracellular ODC target during cell culture work. It would be important to understand whether there are cellular differences in the uptake of DFMO which require higher or lower DFMO levels. The NCI 60-cell line panel using DFMO shows no growth inhibitory effect when used up to 5 mM in any cell line tested. These data are understandable based on the compensatory effect of polyamine uptake on cell growth. Sufficient extracellular polyamine concentrations are available in cell culture media and serum supplements to allow compensation of polyamine needs through uptake by the polyamine transporter after DFMO inhibition of ODC-mediated polyamine biosynthesis.

Clinical oral delivery of AMXT 1501 plus DFMO appears to have reached its limit due to GI tolerability effects.

### Example 2: Safety and Tolerability and Initial Efficacy of Combination of Oral AMXT 1501

Dicaprate with Intravenous DFMO

In order to address the GI intolerability challenge encountered in Part 2 of the clinical trial described in Example 1, a Phase 1B/2A clinical trial is proposed. This study is a multicenter, open-label, Phase 1B/2A dose escalation study, with expansion, designed to evaluate the safety, tolerability, and preliminary efficacy of PO AMXT 1501 in combination with DFMO administered IV in patients with advanced solid tumors, including patients diagnosed with DIPG. The safety, tolerability, preliminary efficacy, and dose escalations for AMXT 1501 plus DFMO will be evaluated following a 3+3 dose escalation design.

In the Phase 1B/2A trial, oral AMXT 1501 dicaprate (e.g., AMXT 1501 dicaprate size 00 capsules described in Example 6) will be administered at a fixed dose of 1,200 mg (free base), split BID, daily, in a fasted state. AMXT 1501 dicaprate will be administered in combination with an escalating dose of continuous IV infusion of DFMO in a four-cohort, [3+3] design (0.5 g, 1 g, 2 g and 4 g/m²) to determine the MTD and RP2D for this combination. DFMO will be dosed using BSA scaling (mg/m²), and escalation will be stopped when average DFMO plasma levels reach 100 μM (ED₉₀). Inclusion criteria include 12-17-year old, juvenile DIPG patients, as well as adults.

An estimated 56 evaluable patients will be enrolled in this study: at least 16 during dose escalation and approximately 40 patients during expansion. The total number of patients will depend on the number of dose levels assessed during dose-escalation to determine the RP2D of PO AMXT 1501 in combination with DFMO administered IV. The primary objective of the study is to determine the safety and tolerability of PO AMXT 1501 dicaprate in combination with DFMO IV in patients with cancer. Secondary objectives include characterization of the PK of AMXT 1501 (administered PO) and DFMO (administered as a continuous infusion), as well as an evaluation of the overall response rate and duration of response. Additional analyses include PD assessments of the impact of AMXT 1501 dicaprate in biopsy tissue and an evaluation of potential biomarkers.

Dramatic improvements in chemotherapeutic drug delivery have recently been made possible using medical device technology. An especially appealing and straightforward technology is offered by Avanos Medical Devices with their elastomer drug infusion pumps. These elastomeric, ambulatory pumps are FDA-approved, and are routinely used for continuous infusions of many types of pharmaceuticals, including pain management agents, oncology agents and antibiotic therapies. No electronics are required. An elastic “ball” is loaded with drug solution under aseptic conditions, and sterile tubing inserts into an infusion port (central catheter) on a patient. A valve in the line regulates the rate of drug infusion (2 mL/hr, or other rates). The system provides drug infusion for up to 5.5 days or longer, and can be easily stopped at any time. Patients may, or may not, require nursing assistance to change the pump with new drug solution, e.g., after 5 days. Pumps with various rates of infusion and drug volumes are available. Alternatively, ambulatory pumps with electronic control can be used. The CADD Legacy electronic pump from ICU Medical is such a pump. An IV bag spike set can be used to provide enough DFMO solution to provide over 7 days of DFMO therapy.

It is expected that continuous IV delivery of DFMO, as by an elastomer infusion pump, would provide sustained, consistent (less variable), and convenient delivery of DFMO for patients, and provide improved PK exposure versus oral delivery. Continuous IV delivery of DFMO is also expected to lower the total amount of DFMO required for patient benefit (e.g., the therapeutically effective dose) in oncology patients, thereby limiting DFMO drug burden and cumulative adverse effects.

For DFMO PK monitoring after continuous intravenous delivery, spot blood sampling would most likely provide reliable drug exposure information (Cₛₛ values). DFMO pharmacokinetics are well understood to predict its behavior, especially for intravenous delivery. It is expected that use of Avanos elastomer drug infusion pumps will greatly reduce variability and GI side effects for DFMO versus oral delivery.

Several DFMO IV dose escalation cohorts are proposed, combined with oral AMXT 1501 dicaprate at 1,200 mg daily, split BID. Specifically, dose-escalation will be conducted across 4 cohorts, starting at 0.5 g/m²/day per day delivered by continuous infusion, for example, in the “ball.” Subsequent cohorts will receive 1 g/m²/day, 2 g/m²/day, and 4 g/m²/day. A 1.18 g/m²/day DFMO dose is expected to achieve ODC target engagement, as indicated by plasma levels of DFMO over 100 μM. By intravenous delivery of DFMO based on patient body surface area (BSA, in m² units), more consistent levels of plasma DFMO can be obtained, in contrast to the variations seen after oral delivery of a fixed dose of DFMO.

Reaching the predicted plasma levels of AMXT 1501 (100 nM) plus DFMO (100 μM) for target engagement is, therefore, the goal in the initial dose-escalation portion of this Phase 1B clinical trial. If no adverse effects are observed at these plasma levels, increased dosages of either or both agents may be warranted.

The trial will involve dilution of stock 200 mg/mL DFMO from sterile vials (20-mL or 50-mL) into ambulatory pumps (Avanos Medical), for delivery of cohort-defined dose levels to patients based on Body Surface Area (BSA, units of m²). The stock 200 mg/mL DFMO sterile vials used are the FDA-approved drug product for trypanosomiasis treatment (Ornidyl).

There are several parameters that are defined initially, including those described below. Choice of the 270 mL Avanos C-Series ambulatory pump (Cat No. C2700020) delivering 2 mL/hr. Therefore, over a 24 h period, 48 mL of DFMO solution is delivered. For a 270-mL pump volume, the maximum delivery duration is 5.625 days. Stock DFMO concentration will be 200 mg/mL in water (identical to FDA-approved Ornidyl formulation). Desired daily DFMO dose levels are described in Table 2.1 and are given in dose levels of mg/m²/day, for the DFMO dose escalation sequence. Among several patient clinical visits, Day 14 is defined as the time for replenishing patients with fresh DFMO filled Avanos pumps. This day coincides with patient clinic visits for routine health and safety monitoring tests. The pumps contain diluted DFMO specific for each individual patient (based on their BSA). Patients will receive three filled pumps during these 14-day clinic visits. Since the total volume delivered over 14 days is 672 mL (14 days×24 hrs×2 mL/hr), and the volume of 3 pumps (810 mL) are nearly identical, pump change-over time will be every 5 days. While at home, the patient, a caregiver or home health expert will change the DFMO pump.

TABLE 2.1
Phase IB DFMO IV Dose Escalation Sequence

| Cohort | mg/m²/day |
|---|---|
| 1 | 500 |
| 2 | 1000 |
| 3 | 2000 |
| 4 | 4000 |

For reference, Ornidyl for trypanosomiasis is delivered IV at a 400 mg/kg/day dose divided QID (four times daily). Ornidyl's package insert directs dilution of 200 mg/mL stock into four parts water for injection (WFI) to obtain an injectable solution within 10% of plasma tonicity. Stock Ornidyl (DFMO) for injection is hypertonic and must be diluted prior to injection for the trypanosomiasis treatment regimen. Package insert directs preparing four injection bags by diluting a total of 100 mL of 200 mg/mL stock into four (25 mL stock each) 100 mL IV dilutant bags. Each bag contains 5 g of DFMO for IV injection over 45 minutes, four times daily (dose amounts based on a 50 kg trypanosomiasis patient). The resulting rate of injection is 125 mL over 45 minutes, therefore, 2.78 mL/min. Diluted solution contains 40 mg/mL for a DFMO injection rate of 111 mg/min. This is a 20× higher DFMO delivery rate than for the highest cohort dose level of 4,000 mg/m²/day (see Table 2.1; for a 2 m² patient, 8000 mg/day, or 5.6 mg/min) in this oncology clinical trial. The slow rate of ambulatory pump delivery (2 mL/hr) makes the hypertonicity issue unimportant for patients in this clinical trial.

The composition of DFMO for infusion in this trial is set forth in Table 2.2.

TABLE 2.2
Composition of DFMO for Infusion 20 mL Vial

| Component | Amount per Vial | Quantity (%) | Function |
|---|---|---|---|
| DEMO HCl H₂O | 4.0 g | 20% | Active Pharmaceutical Ingredient |
| Water for Injection | 16.0 g | 80% | Diluent |
| Total | 20.0 g | 100% | |

Clinical pharmacies will prepare enough diluted DFMO solution to fill three Avanos pumps for the 14-day dispensing interval. Each diluted solution will be prepared based on the specific BSA of the individual patient. A minimum of 810 mL will be required to fill three Avanos pumps. Extra diluted DFMO solution will help facilitate these pump fills. Therefore, a total of 1000 mL of DFMO will be diluted in the clinical pharmacy. This is enough to fill three pumps for 14-day dispensing interval. The dilution scheme works out by diluting 200 mg/mL into 1000 mL, a factor of 5. Equation to solve for Amount (mL) of X Stock DFMO Solution to dilute in 1000 mL of Y mg/mL Diluted Pump Solution: X mL times 200 mg/mL=Y mg/mL times 1000 mL. Solving for X involves multiplying Required Dose Amount (mg) DFMO by 5×. The straightforward dosing amount equation is the following: Desired Dose (mg/m²/day)*BSA gives Required Dose Amount in mg units. Divide by 48 mL daily volume delivered by pump and multiply by 5 to give mL of Stock to add to 1000 mL WFI. An even more straightforward calculation is Desired Dose*BSA*0.10417 (5 divided by 48) gives Volume of 200 mg/mL DFMO stock diluted in 1000 mL Water For Injection (WFI).

This example outlines a straightforward method to calculate dose amounts and dilutions specific for each patient's BSA. Based on several fixed parameters such as pump volume per day and stock DFMO concentration, the amounts of final DFMO dilution can be calculated in the following manner. Example of calculation: Patient BSA (1.8 m²)×Desired Dose amount (500 mg/m²/day)×5/48=X Volume of 200 mg/mL DFMO Stock solution diluted in 1000 mL total Diluted Pump Solution. X=93.75 mL.

### Example 3. Biomarkers for Demonstration of Target Engagement

The clinical plans described in Example 2 also include recruiting patients whose tumors are amenable to biopsy collection and who agree to pre- and during treatment biopsies of said tumors. Examples of cancer indications whose tumors are amenable to biopsy collection include ovarian cancer, thyroid cancer, head and neck cancer, gastric cancer, NSCLC, mesothelioma, esophageal cancer, endometrial cancer, cervical cancer, melanoma, juvenile DIPG, colorectal cancer and breast cancer. For juvenile DIPG patients (aged 12-17), cerebrospinal fluid (CSF) will serve as a biopsy sample (e.g., for AMXT 1501 and DFMO concentration determinations).

Data from literature reporting polyamine levels in different human tumor types suggest tumor polyamine levels of about 100 to 5,000 nmol/g wet tissue should typically be expected. It is believed, based on preclinical results in mouse models, that a meaningful post-treatment sample polyamine decrease (for putrescine (Put) and spermidine (Spd), but not necessarily spermine (Spm)) would be 50% versus pre-treatment sample. Literature polyamine levels are generally reported using units of nmole/g tissue. Use of ‘wet tissue weight’ is a reasonable normalization method. Calculation of the Spd/Spm ratio may be useful in clinical data analyses where spermine levels may serve as an internal control value, as it has been reported that treatment with DFMO or DFMO in combination with AMXT 1501 generally does not impact spermine tumor levels.

For modern LC/MS²-based polyamine determinations, units of ng/g tissue will be used. To convert from nmol/g to ng/g, it is necessary to multiply by the molecular weight (MW) value of the specific polyamine analyte being measured (e.g., Put MW 88.15 g/mole; Spd MW 145.25 g/mole; Spm MW 202.35 g/mole). Therefore, the lower end of the expected tumor polyamine level range of 100-5,000 nmol/g for Put will be 8,800 ng/g; for Spd, it will be 14,500 ng/g and, for Spm, it will be 20,235 ng/g. Upper expectations in tumor concentrations could be 50 times higher than these values. Pre-clinical results using MYCN neuroblastoma mice indicated that values for untreated tumors were generally at or above the higher levels of the expected range. See Gamble, L. D., et al., Inhibition of polyamine synthesis and uptake reduces tumor progression and prolongs survival in mouse models of neuroblastoma. Sci Transl Med. 2019 Jan. 30; 11(477). Putrescine levels are expected to be low: estimates suggest putrescine levels that are 10% of spermidine and/or spermine levels, and this is what was observed in tumors from the MYCN mice.

Generally, the scientific literature has looked at average differences in polyamine-related biomarkers in a number of cancer patients, and compared these to a group of control subjects. The resulting statistical differences were obscured by the high variability of these biomarkers in the populations examined. This results in modest, if at all measurable, statistical differences between group end-points. For this trial, testing AMXT 1501 plus DFMO, the protocol measures differences between pre- and post-treatment matched biopsy pairs. It is expected that the variability between patients when using pre- and post-matched pairs will diminish the intra-patient variability previously observed and reported. The approach to paired sample analysis described here is expected to minimize the variability between different tumor types; the signal will be the degree (e.g., percentage) reduction in polyamine values post-versus pre-treatment. The statistical analysis used will need on-going evaluation, but a comparison between degree of impact (e.g., percent reduction of Put, Spd or Spd/Spm ratio) between the paired samples can be made and statistics on groups of treated patients can be made.

Tumor Drug Levels of AMXT 1501 and DFMO. Expected tumor AMXT 1501 levels are based on pre-clinical distribution studies (4T1 breast cancer (by LC/MS², see Example 5)), and MYCN neuroblastoma murine models (by MALDI, see Example 7)). For clinical assessments, getting corresponding plasma PK levels determinations to gain tumor versus plasma AMXT 1501 and DFMO ratios is important. A plasma target AMXT 1501 level of 1,000 ng/mL has been achieved in three patients in Aminex' First-in-Human trial. Levels were measured in the 4T1 murine model noted above, where tumor AMXT 1501 levels were 894 times higher than plasma AMXT 1501 levels at the 72 h time point. AMXT 1501 tumor levels were near the 1,000 ng/mL level at the 72 h time point. In the MALDI biodistribution study, a 12.7 μM (7,226 ng/mL) level of AMXT 1501 was measured in the MYCN tumors, 24h after AMXT 1501 injection. Target range for clinical tumor biopsy levels at 1,000 ng/g wet tissue level are expected; if higher levels are observed, dilution of samples can be performed. A 100 nM (or 57 ng/mL) is the estimated target engagement concentration for AMXT 1501. Preclinical and clinical data strongly supports the conclusion that AMXT 1501 levels achieved in patients are high enough for full polyamine transporter target engagement.

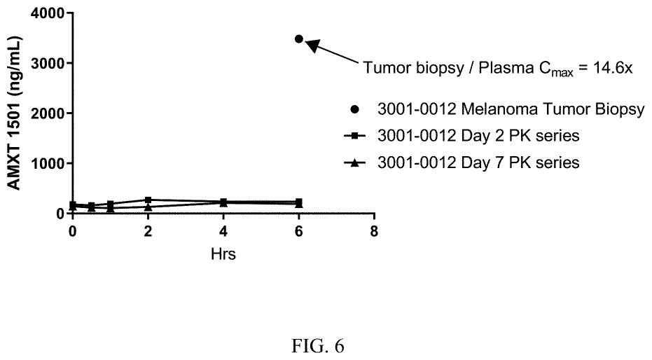

Data supporting the high tumor association of AMXT 1501 has been obtained from a male melanoma patient, treated as part of Aminex's expansion during the initial clinical testing trial. The patient was treated with 1,200 mg daily total AMXT 1501 dicaprate orally (split BID) plus 1,000 mg daily DFMO (split BID) for 18 days, and a tumor biopsy from his scalp was collected. The AMXT 1501 level was 3,480 ng/g or 6.11 μM. Plasma PK levels of AMXT 1501 were also measured for this patient and are shown in FIG. 6. For this patient, the tumor to plasma Cₘₐₓ ratio (3,480 ng/g versus 239 ng/mL) was determined to be 14.6×. These data support tumor AMXT 1501 target engagement at the clinically tolerated dose levels provided to patients. Furthermore, it highlights the high association of AMXT 1501 in tumor tissue, in both human, and mouse tumor settings. Additionally, plasma PK data for n=9 other patients treated at the same dosages showed mean Cₘₐₓ levels of AMXT 1501 of 493 ng/mL+/−339 ng/mL. These data demonstrate a seven-times higher tumor versus plasma Cₘa ratio was obtained. Furthermore, this AMXT 1501 level is above the amount estimated for full target engagement, as discussed above. Further tumor biopsies from AMXT 1501 dicaprate plus DFMO-treated patients are being analyzed. It is expected that these data will be indicative of AMXT 1501 retention in a specific tumor indication and could be used to refine the patient population.

Expected tumor DFMO levels are based on the MYCN pre-clinical distribution study (neuroblastoma (by MALDI)), and were 165-192 μM after overnight drinking of 1% DFMO orally. For DFMO, an expected 100 μM drug level will be needed for engagement of the ODC target (EC₉₀ value). This equates to a 18,200 ng/mL concentration level. The highest clinical DFMO plasma levels observed in the clinical trial described in Example 1 was half this concentration after oral delivery. There is no expectation for increased tumor association of DFMO. Patient tumor concentrations for DFMO are expected to be in the range of 10-100 μM (1,820 to 18,200 ng/mL).

As with reported tumor polyamine levels, only limited, clinical determination of ODC tumor activity has been reported. A gap exists between the clinical testing of DFMO and data supporting its direct inhibition of the drug's target, ODC. Presumably, this is due to the challenging nature of ODC activity measurement (radiolabeled assay), and generally low levels of ODC activities in clinical samples. Nevertheless, as with polyamine tumor levels, data do exist showing higher levels of tumor versus normal tissue ODC activity, which were measurable without stimulation of ODC activity. One report described the use of a label-free, fluorescence-based assay for ODC, which uses a synthetic, off-the-shelf putrescine receptor. Nilam, M., et al. A Label-Free Continuous Fluorescence-Based Assay for Monitoring Ornithine Decarboxylase Activity with a Synthetic Putrescine Receptor. SLAS Discov 22, 906-914 (2017). It is noted that any putrescine in the sample would lead to an initial ‘burst’ of fluorescent signal, which might be an alternative handle on putrescine concentrations. The rate of fluorescence increase following the ‘burst’ would give the desired ODC activity signal. Use of this approach would be expected to generate time-based kinetic data, which may offer an advantage over the single point determinations from the radiolabeled assay technique.

A paper by Garewal et al. provides data on various aspects of clinical ODC activity assessments. Garewal, H. S., Sloan, D., Sampliner, R. E. & Fennerty, B. Ornithine decarboxylase assay in human colorectal mucosa. Methodologic issues of importance to quality control. Int J Cancer 52, 355-358 (1992). Among data provided are: (1) data suggesting phosphate buffer inhibits ODC activity (Tris is recommended); (2) data suggesting need for inclusion of at least 50 g of protein in the assay; and (3) data indicating the importance of inclusion of a DFMO-inhibited blank (10 mM DFMO for 10 minutes). Danzin, C. & Persson, L. L-ornithine-induced inactivation of mammalian ornithine decarboxylase in vitro. Eur J Biochem 166, 45-48 (1987) show the ability of L-ornithine to inhibit ODC activity. It is also well-reported that reducing thiols (e.g., dithiothreitol (DTT)) are important for ODC assay conditions. Janne, J. & Williams-Ashman, H. G. On the purification of L-ornithine decarboxylase from rat prostate and effects of thiol compounds on the enzyme. J Biol Chem 246, 1725-1732 (1971).

Hyvonen, M. T., Keinänen, T. & Alhonen, L. Assay of Ornithine Decarboxylase and Spermidine/Spermine N1-acetyltransferase Activities. Bio-protocol 4, e1301 (2014) describe the radiolabeled assay. These authors advise a pre-assay 0.1 N HCl evaporation step for the radiolabeled substrate 1-¹⁴C-ornithine, and inclusion of a ‘total count’ control in the protocol. Inclusion of a 10 mM DFMO enzyme inhibited control sample, to enable subtraction of “non DFMO-inhibitable counts,” is advisable here as well. This DFMO control sample allows subtraction of any non-specific decarboxylation of ornithine. Essentially, the difference between post-treatment and pre-treatment ODC activity minus total DFMO-inhibitable ODC activity is the relevant value.

ODC activity is normalized to protein content (mg) in the assay. Lowry or Bradford protein determinations are therefore required. A variety of units are used to express ODC activity. Conversion to units of nmol/hr/mg or pmol/hr/mg is suggested.

As with tumor polyamine levels, levels of ODC activity inherent in baseline tumor samples will be used to assess specific tumor indications most susceptible to AMXT 1501 dicaprate plus DFMO therapy. Breast cancers and colorectal cancers are examples of tumors particularly suited to this type of analysis.

Tumor Immunohistochemistry assessment of immune cell infiltration. Specific antibodies and markers to use include H&E, CD3, CD8, FOXP3, CD14, and similar markers for immune cell tumor infiltration for human tumors. Besides the need to define the levels of infiltrating lymphocytes by CD3, CD8, markers for decreased levels of granulocytic MDSC cells are especially important. Human markers for immunosuppressive MDSC granulocytes include CD11b+CD15+CD14−CD33+/loCD66b+ cells. Multiplexing imaging technologies include those offered by Canopy, namely ChipCytometry, or Ultivue, Inc. using FlexVUE technique. Biomarkers associated with these techniques can include CD11b, CD14, PD-L1, CD4, CD68, CD8, PD-1 and CD3.

Gene induction or repression analysis. NanoString immune-profile panel with 770 gene signatures is recommended for tumor biopsies. Standard methods for collection and storage, and analysis of these samples are available (nCounter analysis). A biomarker for IFNγ expression is included in this panel.

The preclinical data from mouse tumor models treated with AMXT 1501 plus DFMO support the expectation of increased tumor infiltrating lymphocytes (TILs) and decreased levels of immunosuppressive MDSC cells. Both these biomarkers of immune stimulation are highly correlated with improved patient outcomes and benefit. The degree of impact on TILs and/or MDSC cell populations are expected to be dependent on the specific tumor type indication studied.

Polyamine-related protein biomarkers by mass spectroscopy. Aminex is developing polyamine-based protein biomarker methods using immunocapture with LC/MS2-based detection. Hypusinated eIF5A (eIF5A-hyp) is a polyamine-derived natural cellular component critical for translational production of proteins involved in cell proliferation. Production of eIF5A-hyp depends on spermidine, which AMXT 1501 plus DFMO has been shown to reduce in cancer cells and mouse tumor models. Translation (production) of the oncogenic proteins MYC and RAS has been shown to be dependent on eIF5A-hypusination and knockdown of this modified protein causes tumor growth arrest. The human eIF5a protein (UniProt P63241) has a mass of 16,832 Daltons and a protein length of 154 amino acids. Hypusination occurs on ⁵⁰Lys and adds 89 Daltons to this protein. LC/MS² identification of Lys50 hypusination of eIF5a is aided by use of LysC peptidase cleavage of the unhypusinated form of the protein. The mature, hypusinated form of eIF5a, is not cleaved by this enzyme at Lys50.

Poulin, R., Lu, L., Ackermann, B., Bey, P. & Pegg, A. E. Mechanism of the irreversible inactivation of mouse ornithine decarboxylase by alpha-difluoromethylornithine. Characterization of sequences at the inhibitor and coenzyme binding sites. J Biol Chem 267, 150-158 (1992) have shown that ODC's cysteine 360 is specifically alkylated by DFMO, producing a unique M+83 S-((2-(1-pyrroline))methyl)cysteine adduct.

Antizyme (AZ) is a natural feedback protein system to reduce cellular polyamines. It does what AMXT 1501 plus DFMO intends to do; inhibits both ODC and polyamine transport. Diminishing AZ protein levels in cells following AMXT 1501 plus DFMO treatment will indicate both agents have engaged their targets. Antizyme Inhibitor (AZIN), also a naturally occurring cellular protein structurally similar to ODC but lacking enzymatic activity, acts to increase polyamine levels by inhibiting the action of AZ by binding more tightly to AZ than to ODC. Upon transformation or stimulation, cells increase the levels of AZIN, together with ODC, to increase polyamine levels required for proliferation. As such, AZIN has oncogene-like properties and its knockdown causes tumor growth inhibition.

Data show these natural polyamine level-modulating systems operate in normal tissues. A fraction of AZ frameshifting occurs normally in cells; depending on their cell cycle needs. With various fractions of cellular ODC dynamically bound to AZ, its impact on polyamine metabolism in relation to cellular growth dynamics is apparent. Furthermore, a fraction of AZ is likewise bound to AZIN. It has also been shown that the majority of cellular eIF5a exists in its mature, hypusinated form.

There is a potential for multiplexing these determinations in one LC/MS² or MALDI run. Evaluation of the impact of AMXT 1501 dicaprate plus DFMO treatment on the polyamine-related proteins listed in Table 3.1 can be explored using Western blot analyses. Furthermore, antibody-based immunoprecipitation and/or isolation can be used in conjunction with LC-MS² analysis to determine protein amounts.

TABLE 3.1
Polyamine-directed biomarker candidates and associated antibodies

| Protein | Protein expression | Antibody |
|---|---|---|
| eIF5A | | Abcam (Ab32443) |
| | Highly expressed in cells and | Abcam (ab227537) |
| | tissues | |
| eIF5A- | Highly expressed in cells and | Millipore (ABS1064) |
| hyp | tissues. | IU-88 (from 21^{st} Century Biochemicals |
| | | Genentech |
| AZ1 | Induced by high polyamines | US Biological O3003-54 - this antibody binds |
| | | both ORF1 and ORF2 AZ1 proteins. |
| AZIN1 | Expressed in proliferating | US Biological 362244 |
| | cells and tissues | |
| ODC | Expressed in proliferating | Ab97395 |
| | cells and tissues. | |
| N-MYC | Amplified in BE(2)-C | Santa Cruz (sc53993) |
| | neuroblastoma cells | |
| BRD4 | Required for expression of | Santa Cruz (sc-518021) |
| | myc | Abcam (EPR5150(2)) |

### Example 4: Ex Vivo Blood-based Pharmacodynamic (PD) Bioassay of Polyamine Transport Inhibition

To evaluate the target engagement activity of AMXT 1501 after oral administration in human patients, an ex vivo PD assay assessing lymphocyte spermine uptake and inhibition of uptake by AMXT 1501 was developed. Briefly, uptake of a fluorescent-labeled spermine analog conjugate (Fl-Spm, Compound 5, C5, Annereau, J. P., et al., A fluorescent biomarker of the polyamine transport system to select patients with AML for F14512 treatment. Leuk Res. 2010 October; 34(10):1383-9) was assayed by fluorescence-activated cell sorting (FACS) analysis in CD4+ and CD4⁻ T cells in human blood samples stimulated with anti-CD3+ and anti-CD28+ antibodies. In whole human blood samples treated with 0-2,000 nM AMXT 1501, maximal fluorescent-labeled spermine uptake inhibition in the absence of DFMO occurred at 250 nM AMXT 1501 with an ED₅₀ value of approximately 75 nM. Results from these in vitro-ex vivo experiments demonstrated that AMXT 1501 added to whole human blood robustly engaged and inhibited polyamine uptake into activated lymphocytes.

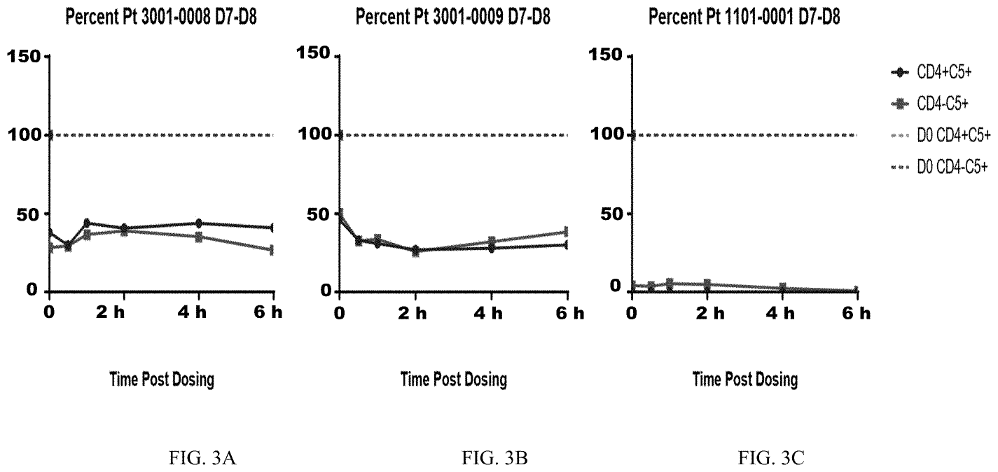

The assay was used to evaluate target engagement activity of AMXT 1501 samples from patients orally dosed with AMXT 1501 dicaprate in enterically-coated capsules according to the clinical trial protocol described in Example 1. Polyamine uptake in blood lymphocytes (CD4⁺ or CD8⁺ T cells) was measured at the same time points as the PK levels for AMXT 1501 were measured. Comparisons were made between uptake in the pre-treatment sample and samples taken over time following dosing. Preliminary results showed sustained AMXT 1501 target engagement activity in several Part 2, Cohort 3 patients' whole blood samples. FIGS. 3A, 3B and 3C show inhibition of C5 uptake in Day 7-8 ex vivo patient blood CD4+ and CD4-lymphocytes from Part 2, Cohort 3 patients treated at oral RP2D level of AMXT 1501 dicaprate.

These experiments do not directly demonstrate AMXT 1501 target engagement in patients' tumors, but do show AMXT 1501 present in patient blood samples after PO dosing is active, and inhibits T cell uptake ex vivo after activation and proliferation stimulation. These PD assays demonstrated that the AMXT 1501 in circulation is functional, with clinical AMXT 1501 PK concentrations consistent with those shown to be efficacious in mouse cancer models.

Pharmacodynamic Bioassay. The purpose of this whole blood assay was to quantify the inhibition of polyamine transport in T cells in human subjects receiving AMXT 1501. Collected whole blood from subjects receiving AMXT 1501 was diluted with serum-free media and stimulated with anti-CD3 (T cell receptor engagement) and anti-CD28 (co-stimulatory signal) to drive T cell activation and proliferation for a total of three days. During the last 12-18 hours of culture, a fluorescently-labeled spermine analog (Fl-Spm, Compound 5, C5) was added to the in vitro culture to monitor polyamine uptake into cells. The functional activity (e.g., inhibitory effect) of AMXT 1501 on C5 uptake by T cells was then determined using flow cytometry by measuring the frequency and median fluorescence intensity (MFI) of C5 in T cells. Activation and proliferation of T cells is required for the uptake of C5; however, in the presence of high concentrations of AMXT 1501 in vitro, C5 uptake is inhibited despite active proliferation of T cells. Therefore, a whole blood sample spiked with high levels of AMXT 1501 prior to culture will be used as a positive control for each subject in this assay.

A BD FACS Canto II with the long-pass (LP) mirrors and bandpass (BP) filters detailed in Table 4.1 was used in the development and optimization of this method.

TABLE 4.1

| Laser | BP Filter or LP Mirror | LP Mirror | Fluorochrome/Dye |
|---|---|---|---|
| Blue (488 nm) | 670 LP | 655 LP | PerCP-Cy5-5 |
| | 530/30 BP | 502 LP | FITC/Compound 5 |
| | 488/10 BP | | SSC |
| Red (633 nm) | 780/60 BP | 735 LP | APC-H7 |
| Violet (405 nm) | 510/50 BP | 502 LP | BV 510/Aqua |
| | 450/50 BP | | BV421 |

An incubator at 95% humidity, 37° C. and 5% CO₂ was used. The reagents in Table 4.2 were also used.

TABLE 4.2

| | | Catalogue | |
|---|---|---|---|
| Reagent | Manufacturer | Number | Storage |
| Purified NA/LE Mouse Anti-Human CD3 | BD Pharmingen | 555329 | 2-8° C. |
| (Clone UCHT1) | | | |
| Purified NA/LE Mouse Anti-Human CD28 | BD Pharmingen | 555725 | 2-8° C. |
| (Clone 28.2) | | | |
| AIM V Medium | Gibco | 12055-083 | 2-8° C. |
| Dulbecco's phosphate-buffered saline (DPBS) | Gibco | 14190-144 | RT |
| LIVE/DEAD Fixable Aqua Dead Cell Stain | Molecular | L34957 | ≤−20° C. |
| | Probes | | |
| Distilled water (dH₂O) | Sigma | W3500 | RT |
| BD Pharm Lyse Lysing Buffer (10X | BD Bioscience | 555899 | 2-8° C. |
| concentrate) | | | |
| BD FACS Lysing Solution (10X concentrate) | BD Bioscience | 349202 | 2-25° C. |
| BD Perm/Wash buffer | BD Bioscience | 554723 | 2-8° C. |
| BD Stain Buffer (FBS) | BD Pharmingen | 554656 | 2-8° C. |
| BD Stabilizing Fixative (3X concentrate) | BD Bioscience | 339860 | RT |
| Anti-Mouse Ig, κ/Negative Control | BD Bioscience | 552843 | 2-8° C. |
| Compensation Particles Set | | | |

The antibodies in Table 4.3 were also used.

TABLE 4.3

| | | | | Catalogue | |
|---|---|---|---|---|---|
| Antigen | Fluorochrome | Clone | Manufacturer | Number | Storage |
| CD3 | APC H7 | SK7 | BD Biosciences | 560176 | 2-8° C. |
| CD4 | PerCP-Cy5.5 | RPA- | BD Biosciences | 560650 | 2-8° C. |
| | | T4 | | | |
| Ki67 | Brilliant Violet (BV) 421 | B56 | BD Biosciences | 562899 | 2-8° C. |
| Any | FITC (Use as a | | | | |
| | compensation control for | | | | |
| | Compound 5) | | | | |
| Any | BV510* (Use as a | | | | |
| | compensation control for | | | | |
| | LIVE/DEAD Aqua | | | | |
| | Stain) | | | | |

*Alternatively, ArC Amine Reactive Compensation Beads stained with LIVE/DEAD Aqua stain can be used as a compensation control.

Reagent/Buffer Preparation
Purified NA/LE Mouse Anti-Human CD28 (Clone 28.2): Stock concentration 1 mg/ml. Final concentration per reaction: 1 μg/ml. Prepared a 100 μg/ml working dilution by diluting stock 1:10 with AIM-V. Volume of 100 μg/ml of anti-CD28 required per subject (5× culture conditions)=30 μl.
Compound 5: Prepared a working solution of 1 mM, as per manufacturer's instructions. A working dilution was freshly prepared on a weekly basis. Final concentration per reaction: 4 μM. Kept working dilution at 4° C. until required. Prepared solutions were protected from light exposure.
AMXT 1501: Prepared a working solution of 100 μM, as per manufacturer's instructions. A working dilution was freshly prepared on a weekly basis. Final concentration of spiked control: 2 μM. Kept working dilution at 4° C. until required.
LIVE/DEAD Fixable Aqua Dead Cell Stain: Prepared stock concentration as per manufacturer's instructions and stored at ≤−20° C. Prepared working dilution prior to staining cells by diluting stock 1:1000 in DPBS. Kept working dilution at 4° C. until required for staining. Stock and working dilutions were protected from light exposure. Working dilutions were freshly prepared prior to staining. Volume of working dilution of LIVE/DEAD Fixable Aqua Dead Cell Stain required per subject (5× culture conditions)=1250 μl.
1×BD Pharm Lyse Buffer: Diluted the 10× concentrate 1:10 with dH₂O. Volume of 1× Pharm Lyse required per subject (5× culture conditions)=15 ml. 1× diluted solutions may be stored at 4° C. for up to 30 days.
1×BD FACS Lysing Solution: Diluted the 10× concentrate 1:10 with room temperature (RT) (20° C.-25° C.) dH₂O. Volume of 1×FACS Lysing Solution required per subject (5×culture conditions)=10 ml. The prepared solution is stable for one month when stored in a glass or high-density polyethylene (HDPE) container at RT.
1×BD Perm/Wash buffer: Diluted the 10× concentrate 1:10 with dH₂O. Volume of 1×BD Perm/Wash required per subject (5× culture conditions)=5 ml. 1× diluted solutions may be stored at 4° C. for up to 30 days.
1×BD Stabilizing Fixative: Diluted the 3× concentrate 1:3 with dH₂O. Volume of 1×BD Stabilizing Fixative required per subject (5× culture conditions)=1250 μl. 1× diluted solutions are stable for one month when stored in a polystyrene container at 2° C.-8° C.

Method. Lithium-heparin vacutainers containing whole blood from subjects were stored upright at room temperature. In vitro culture was set up within 12 hours of collecting blood. Prior to setting up of in vitro culture, AIM-V media was warmed to 37° C. in a water bath or incubator. Five FACS tubes per subject were labelled as per conditions outlined in Table 4.4. Appropriate volumes of whole blood, AIM-V media and reagent(s) were added to each tube as outlined in Table 4.4. Prior to aliquoting blood, ensured that blood was mixed well by inverting the vacutainer several times.

TABLE 4.4

| | | | | Anti- | AMXT | |
|---|---|---|---|---|---|---|
| | | Whole | Anti-CD28 | CD3 | 1501 | |
| Condition | Description | blood | (100 μg/ml) | (1 mg/ml) | (100 μM) | AIM-V |
| Media only | Negative control | 200 μl | | | | 800 μl |
| Stim only | Proliferation control | 200 μl | 10 μl | 10 μl | | 780 μl |
| C5 only | C5 gating control | 200 μl | | | | 800 μl |
| AMXT 1501 | AMXT 1501 control | 200 μl | 10 μl | 10 μl | 20 μl | 770 μl |
| spiked | | | | | | |
| control + C5 | | | | | | |
| Stim + C5 | To determine the | 200 μl | 10 μl | 10 μl | | 780 μl |
| | functional activity of | | | | | |
| | in vivo AMXT 1501 | | | | | |
| | levels in subjects | | | | | |

Gently vortexed tubes and placed tubes in an incubator at 37° C. in 5% CO₂ and 95% humidity. The FACS tubes had a dual snap-cap enabling both culturing and storage of samples in the same tube. Left the cap loose to allow for aerobic culturing of cells. After 48 hours of incubation, removed tubes from incubator and added C5 to the conditions detailed in Table 4.5.

TABLE 4.5

| | | Compound 5 |
|---|---|---|
| Condition | Description | (1 mM) |
| Media only | Negative control | None |
| Stim control | Proliferation control | None |
| Compound 5 | C5 gating control | 4 μl |
| AMXT 1501 | AMXT 1501 control | 4 μl |
| control + C5 | | |
| Stim + C5 | To determine the functional activity of | 4 μl |
| | in vivo AMXT1501 levels in subjects | |

Gently vortexed tubes and placed in an incubator at 37° C. in 5% CO₂ and 95% humidity for 12-18 hours.

Staining of samples. Prepared 1× Pharm Lyse solution with dH₂O and warmed to 37° C. in a water bath or incubator. Prepared a working dilution of LIVE/DEAD Aqua Stain and stored at 4° C. until required. Protected from exposure to direct light. Removed samples from incubator and added 3 ml of 1× Pharm Lyse to each tube, gently vortexed tubes and incubated tubes for 15 minutes at RT. Following the incubation step, pelleted cells by centrifugation at 400×g for 5 minutes at RT. Discarded supernatant from each tube and carefully blotted the tube on a paper towel. Washed cells by adding 2 mL of DPBS to each tube, and resuspended the cell pellet by gently pipetting cells up and down. Please note that red blood cell (RBC) contamination may still be present at this stage but will be lysed during the fixation/lysis step with BD FACS Lysing Solution. Pelleted cells by centrifugation at 400×g for 5 minutes at RT. Discarded supernatant from each tube and carefully blotted the tube on a paper towel. Stained cells with LIVE/DEAD Aqua stain by adding 250 μl of the diluted LIVE/DEAD Aqua Stain (working dilution) to cells, mixing the resulting mixture and incubating the cells for 20 minutes at RT in the dark. Please note that once cells are stained with LIVE/DEAD Aqua stain, samples should always be protected from direct exposure to light. Following the incubation step, added 2 ml DPBS to each tube and pelleted by centrifugation at 400×g for 5 minutes at RT. Discarded supernatant from each tube and carefully blotted the tube on a paper towel. Resuspended the cell pellet in 2 mL BD FACS Lysing Solution and incubated for 10 minutes at RT in the dark. Pelleted cells by centrifugation at 400×g for 5 minutes at RT. Discarded supernatant from each tube and carefully blotted the tube on a paper towel. Washed cells by resuspending the pellet in 2 ml DPBS and pelleted by centrifugation at 400×g for 5 minutes at RT. Discarded supernatant from each tube and carefully blotted the tube on a paper towel. Resuspended cells in 1 mL 1×BD Perm/Wash and incubated at RT for 10 minutes in the dark. Pelleted cells by centrifugation at 400×g for 5 minutes at RT. Discarded supernatant from each tube and carefully blotted the tube on a paper towel. Calculated the volume of antibodies required to stain all sample tubes (5 tubes per subject) using Table 4.6. Kept antibody stock vials on ice and always protected antibodies from exposure to direct light.

TABLE 4.6
Antibody staining master mix table

| | Volume per tube | Total number of tubes: _ |
|---|---|---|
| CD3 APC-H7 | 2.5 μl | |
| CD4 PerCP-Cy5.5 | 2 μl | |
| Ki67 BV410 | 2.5 μl | |
| 1 × BD Perm/Wash | 13 μl | |
| Total Volume | 20 μl | |

Added 20 μl of the antibody master mix to each tube, gently vortexed samples and stained cells for 20 minutes at RT in the dark. Following the incubation step, added 2 ml BD Stain Buffer to each tube and pelleted by centrifugation at 400×g for 5 minutes at RT. Discarded supernatant from each tube and carefully blotted the tube on a paper towel. Resuspended cells in 250 μl of 1× Stabilization Buffer. Stained samples were protected from light (wrapped in foil) and stored at 2-8° C. until acquisition. Samples were acquired within four hours of staining.

Preparation and acquisition of compensation controls. Vortexed BD CompBeads thoroughly before use. Labelled separate FACS tubes for each fluorochrome-conjugated mouse Ig, x antibody used in the master mix. Please note that CompBeads stained with a BV510 conjugate were used as a compensation control for Aqua and that a FITC conjugate was used as a compensation control for Compound 5. Alternatively, ArC Amine Reactive Compensation Beads stained with LIVE/DEAD Aqua stain can be used as a compensation control. Added one full drop (approximately 60 l) of the BD CompBeads Negative Control and one drop of the BD CompBeads Anti-Mouse Ig, x beads to each tube. Added the appropriate volume of antibody (listed below) to the appropriately labeled tube, ensuring the antibody was deposited to the bead mixture, then vortexed.
FITC—Titer recommended by manufacturer (spermine compensation)
BV510*—Titer recommended by manufacturer (Aqua compensation) *Alternatively, ArC Amine Reactive Compensation Beads stained with LIVE/DEAD Aqua stain can be used as a compensation control.
APC-H7—2.5 μl
PerCP-Cy5.5—2 μl
BV410—2.5 μl

Incubated beads for 15-30 minutes at RT. Protected from exposure to direct light. Following incubation, added 2 ml Stain Buffer to each tube and pelleted by centrifugation at 200×g for 10 minutes. Discarded supernatant from each tube and carefully blotted the tube on a paper towel. Resuspended bead pellet in each tube by adding 250 μl of Stain Buffer to each tube. Vortexed thoroughly. Ran each tube separately on the flow cytometer. Gated on the singlet bead population based on FSC (forward-light scatter) and SSC (side-light scatter) characteristics, and acquired at least 10,000 bead events. After acquiring each compensation control, verified that the snap-to gates were appropriately placed over the positive bead peak and created an auto-interval gate around the negative population in each fluorescence histogram. Repeated this step for remaining compensation tubes, as necessary. After compensation data had been recorded and gates had been adjusted, calculated compensation. If the compensation calculation was successful, “Link & Save” the compensation set-up to the experiment. Proceeded to acquire the stained subject samples.

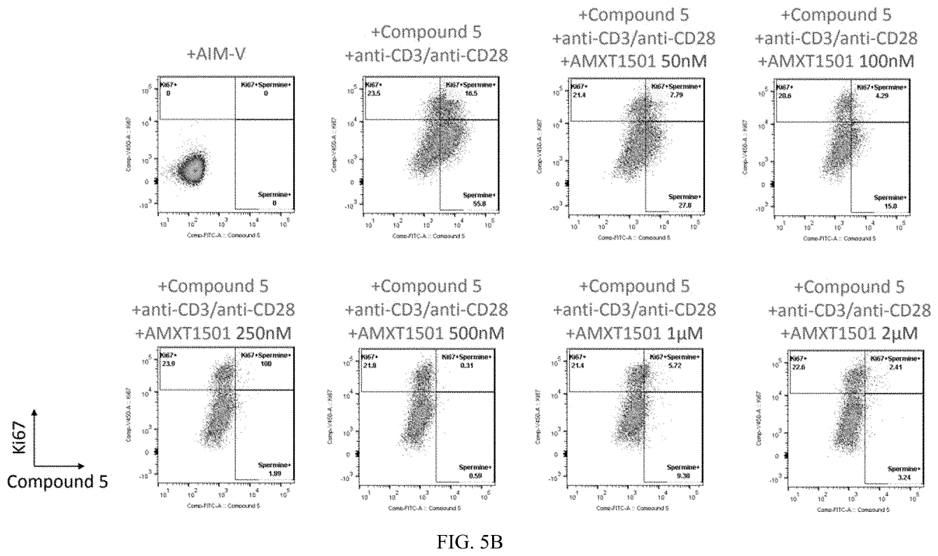

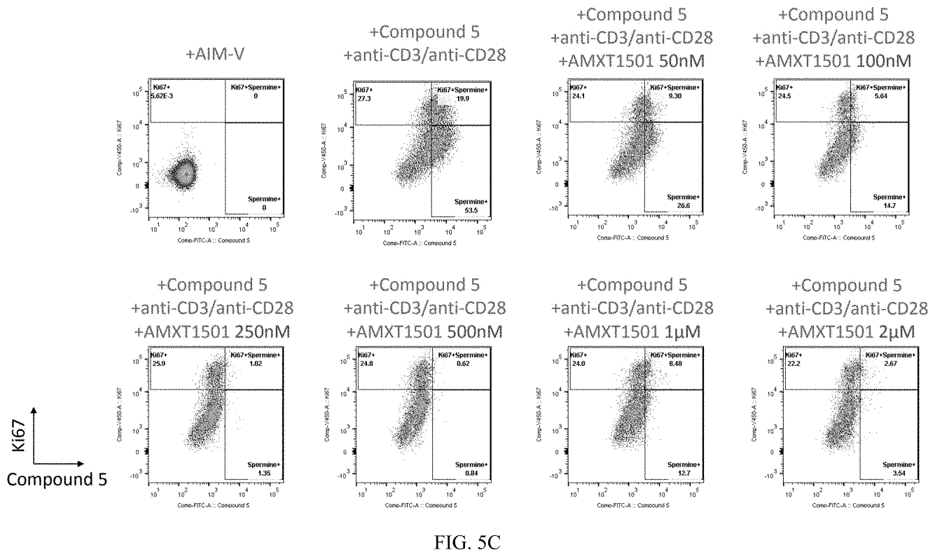

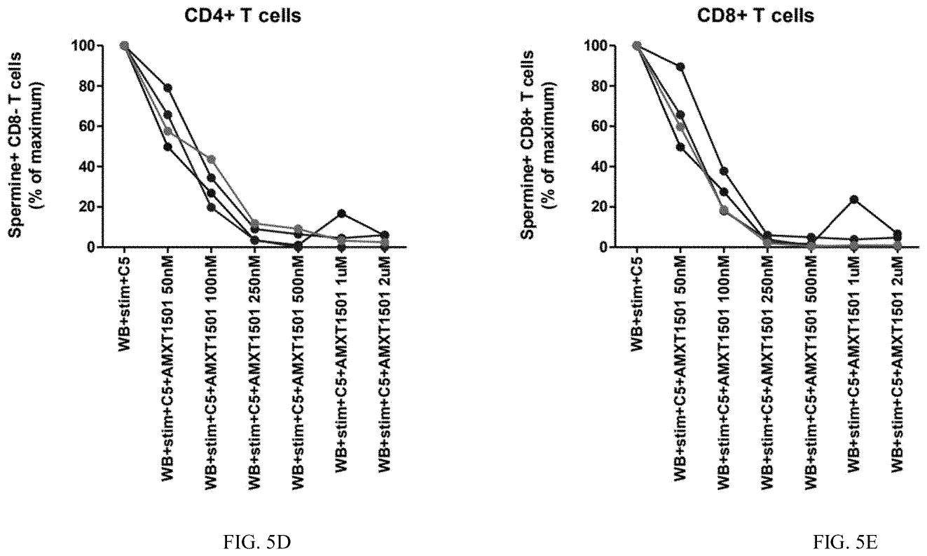

Acquisition of subject samples. Adjusted FSC and SSC voltages appropriately to display the lymphocyte population and included an appropriate threshold on FSC to exclude debris. For the stained subject samples, at least 50,000 live CD3+ events were acquired using the gating strategy depicted in FIG. 5A. First, gated on the lymphocyte population based on FSC and SSC characteristics. From the lymphocyte population, gated on live CD3+ T using a LIVE/DEAD Aqua versus CD3 dot plot. The lymphocyte gate and live CD3 gate needed to be modified between samples with no proliferation (no anti-CD3/anti-CD28 stimulation, whole blood+AIM-V (negative control)), and samples in which T cell proliferation had occurred to include blasting T cells (whole blood+anti-CD3/anti-CD28).

FIGS. 5B and 5C show titration of AMXT 1501 for inhibition of C5 uptake into CD4 lymphocytes and CD8 lymphocytes, respectively, from whole blood. FIGS. 5D and 5E provide a summary of the data in FIGS. 5B and 5C, respectively.

### Example 5: Tumor Pharmacokinetics and Tissue Distribution By LC/MS²

In a tumor tissue distribution study, groups of 4T1 breast tumor-bearing BALB/c mice were treated with a single dose of AMXT 1501 4HCl (2.5 mg/kg SC) with or without DFMO at 400 mg/kg PO. DFMO treatment was given QD for three days, with AMXT 1501 given three hours after the last PO DFMO dose. Tumors and tissues were harvested at various time points after SC injection of AMXT 1501 4HCl, and analyzed for AMXT 1501 content. All bioanalytical AMXT 1501 concentration assessments utilized LC/MS/MS and are based on the plasma, tissue, and tumor AMXT 1501 free base content.

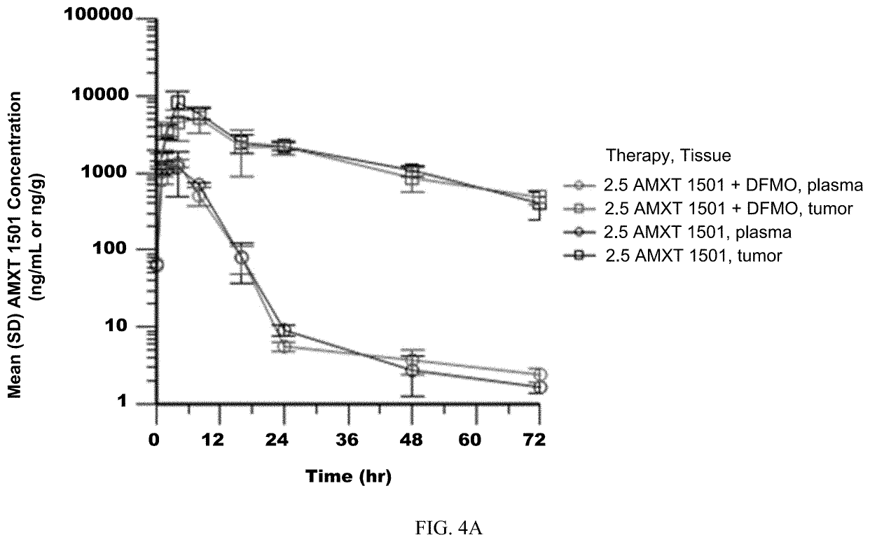

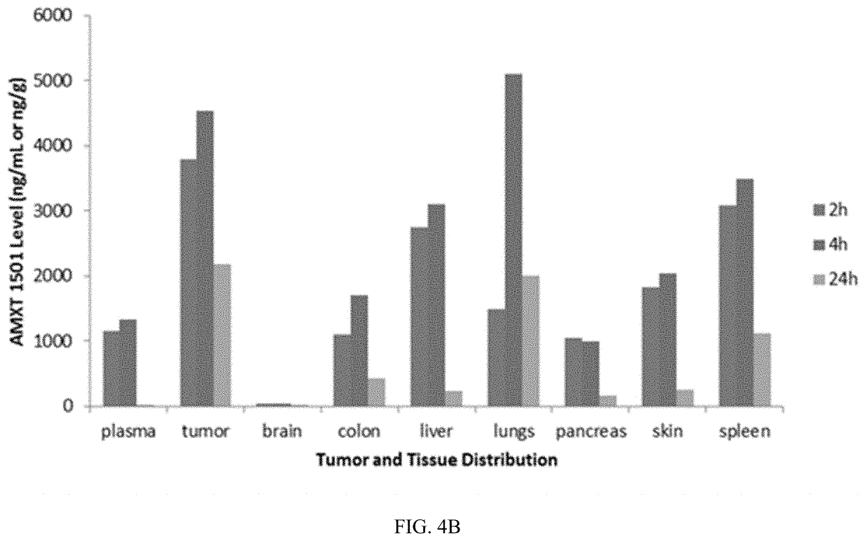

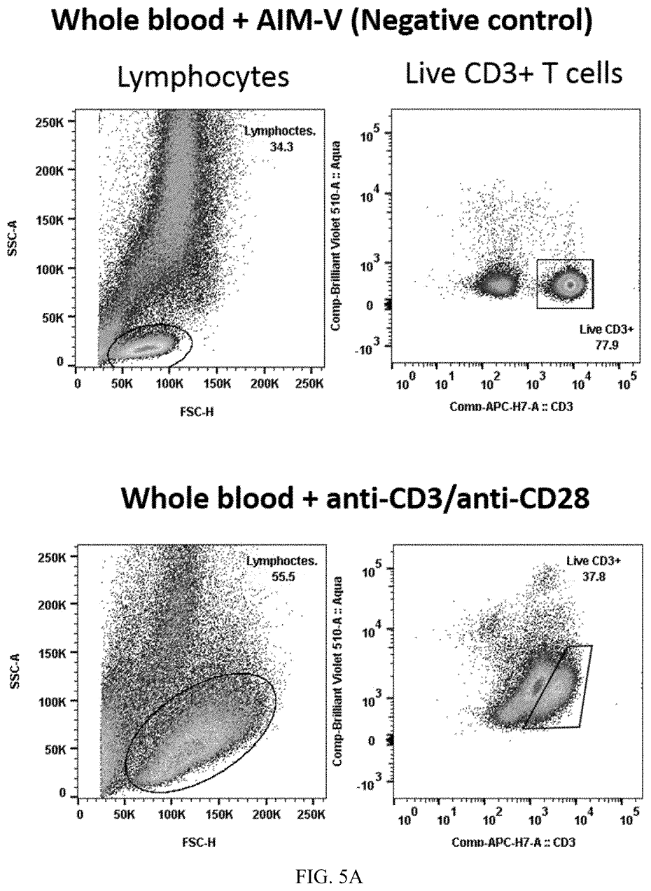

FIG. 4A shows tumor pharmacokinetics and retention of AMXT 1501 in tumor and plasma with or without DFMO co-treatment. The data in FIG. 4A show a high tumor localization and retention of AMXT 1501 following SC administration in comparison to plasma. Once AMXT 1501 associated with the tumor, its elimination appeared to follow prolonged kinetics compared to elimination from blood, and tumor association after a single dose is much higher versus blood. Substantial drug was retained by the tumor even three days following a single injection of 2.5 mg/kg AMXT 1501 4HCl. At 72 hours after SC injection, the tumor/plasma AMXT 1501 ratio was 894. These data suggest a 20- to 100-fold increase in AMXT 1501 concentration over the in vitro average EC₅₀ values for tumor cell growth inhibition were obtained. Furthermore, a 15.5× higher tumor/plasma AUC_{0-∞} value (138,000 versus 8,890 hr*ng/mL) was observed in this study. These data strongly suggest that treatment with AMXT 1501 dicaprate will result in high retention in tumors to provide adequate drug exposure in the tumor.

A series of seven tissues were also collected to quantify the tissue distribution of AMXT 1501 (brain, colon, liver, lung, pancreas, skin, and spleen) following SC administration. Tissues were collected at three time points (2, 4, and 24 hours) and plasma, tumor, and tissue levels of AMXT 1501 were determined by LC/MS/MS. FIG. 4B shows the level of AMXT 1501 in plasma, tumor and the seven collected tissues at 2, 4 and 24 hours post-administration of AMXT 1501. The data in FIG. 4B demonstrate the expected tumor selectivity for AMXT 1501.

Data from this study showed DFMO had no effect on AMXT 1501 tumor retention.

### Example 6: Preparation of Enterically-Coated Capsules Containing AMXT 1501 Dicaprate

This example describes the use of CAPSUGEL® VCAPS® enterically-coated capsules. Size 00 capsules were used to prepare AMXT 1501 dicaprate capsules containing 200 mg AMXT 1501, based on free base content. To calculate the amount of AMXT 1501 dicaprate required to encapsulate this amount of free base content, the salt conversion factor of 1.60× should be used (AMXT 1501 dicaprate MW 913.47 g/mol divided by AMXT 1501 free base MW 569.09 g/mol). Therefore, about 320 mg weight of AMXT 1501 dicaprate per capsule is required. A variety of manual, semi-automated or automatic equipment and techniques can be used to encapsulate AMXT 1501 dicaprate. For this example, a ProFiller 3700 encapsulator by Torpac was employed. For capsules for clinical use, full cGMP procedures were followed. Approximately 97.0 g of sieved cGMP AMXT 1501 dicaprate powder was weighed into a suitably sized weigh boat, and filled into 300 empty capsules using the ProFiller with 300-hole trays. The filled capsules were then closed with capsule snap caps using the ProFiller device. The resulting filled capsules were polished, sized to ensure closure and stored refrigerated for packaging.

Table 5 shows the composition of the resulting drug product.

TABLE 5
Composition of AMXT 1501 Dicaprate Size 00 Capsules

| | Unit Formula | Quantity |
|---|---|---|
| Component | (mg/capsule) | (%) |
| AMXT 1501 Dicaprate | 317.8ª | 100.00 |
| Capsule Shell, Size 00^{b} | 125.8 | — |
| Total | 443.6 | 100.0 |

^{a}Equivalent to 200 mg active free base.
^{b}Composed of hypromellose, hypromellose acetate succinate, ammonium hydroxide, and titanium dioxide.

Filled AMXT 1501 dicaprate capsules were packaged in 30-count bottles for clinical use, e.g., in the clinical trial described in Example 1 and/or Example 2. HDPE bottles (100 cc) with induction seals, child-proof SecuFx caps and desiccant canisters (containing 2 g silica gel) were used. Filled bottles were labeled and stored at refrigerated temperatures (2-8° C.) until use.

### Example 7: Distribution and Quantification of DFMO, AMXT 1501, and PD Markers Putrescine, Spermidine and Spermine By MALDI-FT ICR Imaging Technology

The aim of the study was to follow DFMO, AMXT-1501, and PD markers putrescine, spermidine and spermine in mouse whole-body sections in order to understand the efficacy of the mono and combination therapies.

Test System. Male Th-MYCN mice, approximately 5-6 weeks of age, with spontaneous neuroblastoma tumor development commenced treatment according to Table 6.1 when tumors were approximately 5 mm in size. AMXT 1501 dicaprate (free base content) delivery was by SC (subcutaneous) daily injections, and DFMO was ad libitum in drinking water. Approximate intake of DFMO over 96 hours is 160 mg (40 mg/day). AMXT 1501 dicaprate was dissolved in a buffered mannitol solution and pH adjusted to between 6.2-6.5 for administration. Time point: three treatments, cull 24 hours after last treatment in the morning.

TABLE 6.1
Administration Conditions

| Test | | | AMXT 1501 | Administration | Time Point |
|---|---|---|---|---|---|
| Subject | Type | DFMO (%) | (mg/kg) | Mode | (hours) |
| 001 | Control | Vehicle Only | | SC | 96 |
| 018 | Control | Vehicle Only | | SC | 96 |
| 049 | Treated | 1% | n/a | 96 hours | 96 |
| 082 | Treated | 1% | n/a | 96 hours | 96 |
| 105 | Treated | n/a | 2.5 | 96 hours | 96 |
| 107 | Treated | n/a | 2.5 | 96 hours | 96 |
| 284 | Treated | 1% | 2.5 | 96 hours | 96 |
| 286 | Treated | 1% | 2.5 | 96 hours | 96 |

Sample Preparation. Animals were euthanized on the morning of the 4th day, after three days of treatment and shaved, and then the carcasses were frozen. Tissues stored at −80° C. were placed inside the cryostat. The temperature of the cryostat was maintained around −20° C. and −40° C. 20-am tissue sections in the sagittal plane going through the left eye were obtained for each carcass. Tissue sections were mounted directly on indium tin oxide (ITO) slides.

Tissue sections on ITO slides were placed in a desiccator for 15 minutes before matrix deposition. An image of the slides was then acquired with a scanner to synchronize positions of the tissue sections with the target of the laser.

To prepare stock solutions: DFMO was first dissolved in water at 15 mM, and aliquoted prior to further dilution in water; AMXT 1501, putrescine, spermine, and spermidine were first dissolved in water at 10 mM and aliquoted prior to further dilution in water.

Dilution series of the test items were prepared in water from the stock solutions. For quantification purposes, the different concentrations of the drug were deposited as follows: one microliter of each point was spotted on ITO slide for putrescine, spermine, and spermidine or control liver tissue for DFMO and AMXT 1501. A spot of pure solvent was added as a negative control. Slides were then placed in a desiccator for 15 minutes before MALDI matrix deposition. The control liver tissue consisted of a liver homogenate spiked with an olanzapine standard at 40 g/g of tissue. Sections of the liver homogenate were prepared and added on each ITO slide in order to monitor the signal of the FTICR through the different acquisitions and thus validate the comparison between slides.

For AMXT 1501, 2,5-dihydroxybenzoic acid (DHB) at 40 mg/ml in 1:1 MeOH/H₂O with 1% trifluoroacetic acid (TFA) and 25 nM of ¹³C4-AMXT 1501 was sprayed on the samples with an automatic sprayer (TM-Sprayer, HTX-Imaging). DFMO, putrescine, spermine, and spermidine samples were chemically derivatized for 30 minutes prior to matrix deposition. DHB at 40 mg/ml in 7:3 MeOH/H₂O with 0.1% TFA and 5 μM each of chemically derivatized D₃-DFMO, [¹³C₂,¹⁵N₂]-putrescine, ¹⁵N₃-spermidine, and [¹³C₂,¹⁵N₂]-spermine was sprayed on the samples with an automatic sprayer (TM-Sprayer, HTX-Imaging).

The mass spectrometry imaging (MSI) acquisitions were performed using the parameters in Table 6.2 with a data reduction parameter set at 95%. MSI data were acquired and analyzed with FlexImaging 4.1 (Bruker Daltonics), Data Analysis 4.0 (Bruker Daltonics) and Multimaging™ 1.2.2 (ImaBioTech).

TABLE 6.2
MALDI-FTICR Acquisition Parameters

| | | DFMO | |
|---|---|---|---|
| | | Spermine | |
| | AMXT 1501 | Spermidine | Putrescine |
| Mode | CASI | CASI | CASI |
| Ionization | Positive | Positive | Positive |
| Mass Range | 560 ± 15 kDa | 370 ± 45 kDa | 287 ± 15 kDa |
| Laser Frequency | 2,000 Hz | 2,000 Hz | 2,000 Hz |
| Calibration Mode | 2 | 2 | 2 |
| Spatial Resolution | 350 μm for the entire whole-body sections | | |
| | 200 μm spatial resolution for the calibration range | | |

Intensity scales representing the molecular signal were adjusted for each image to discriminate the noise from the molecular signal but also to give the best visualization of the signal across the sections. The normalization was based on the signal of the internal standard ¹³C4-AMXT 1501 (for AMXT 1501) or D₃-DFMO, ¹⁵N₃-spermidine, or [¹³C₂,¹⁵N₂]-spermine (for DFMO/spermidine/spermine). Convolution step was performed on the original images using a normalized uniform kernel, which averages the values around a position. The kernel size was manually optimized for the analysis, minimizing the background noise. Based on the MSI data set on the tissue sections and the dilution series, the quantitation of the test items was performed with the IS approach. A correlation between the calibration curve and the signal obtained on the tissues was performed in order to determine the concentration of the test items per histological structure in g/g of tissue and μM.

Distribution of AMXT 1501. AMXT 1501 has been imaged in mice treated with either AMXT 1501 alone, or in combination with DFMO. Images of vehicle-treated animals did not reveal the presence of any interfering signal, demonstrating the specificity of the analytical method. In AMXT 1501 only-treated mice, the drug showed a wide exposure, especially in the lung, thymus, spleen, kidney and tumor. Other tissues such as liver, brown and white fat, stomach and intestine contents, presented a lower exposure to AMXT 1501. Skin and CNS did not show a significant exposure to AMXT 1501. Bile ducts are not visible at the spatial resolution used for this whole-body experiment. In mice receiving the combination therapy, lung was again the organ showing the highest AMXT 1501 exposure.

Concentrations of AMXT 1501 measured in mice treated with AMXT 1501 alone ranged from 3.36 to 17.01 μg/g, from intestine contents to lung, respectively (Table 6.3).

TABLE 6.3
Quantitation of AMXT 1501 in μg/g in the whole-body section

| Treatment | Vehicle | | DFMO | | AMXT-1501 | | COMBO | |
|---|---|---|---|---|---|---|---|---|
| Animal ID | 001 | 018 | 049 | 082 | 105 | 107 | 284 | 286 |
| Brain | ND | ND | NA | NA | ND | ND | NA | ND |
| Brown fat | ND | ND | NA | NA | 10.5 | ND | 8.33 | 8.11 |
| Cardiac blood | ND | ND | NA | NA | ND | ND | 8.37 | 8.03 |
| Eye | ND | ND | NA | NA | ND | BLOQ | 9.30 | ND |
| Intestine contents | ND | ND | NA | NA | 9.02 | 3.36 | 8.47 | 8.10 |
| Intestine wall | ND | ND | NA | NA | 11.4 | BLOQ | 9.00 | 8.06 |
| Kidney | ND | ND | NA | NA | 12.1 | ND | 5.64 | ND |
| Liver | ND | ND | NA | NA | 9.53 | BLOQ | 8.87 | 7.45 |
| Lung | ND | ND | NA | NA | 17.0 | 10.27 | 14.5 | 10.0 |
| Myocardium | ND | ND | NA | NA | 9.59 | ND | 8.44 | 8.30 |
| Pancreas | ND | ND | NA | NA | 8.54 | ND | 8.43 | ND |
| Spleen | ND | ND | NA | NA | 12.9 | ND | NA | ND |
| Stomach contents | ND | ND | NA | NA | 8.48 | BLOQ | 8.80 | ND |
| Stomach wall | ND | ND | NA | NA | 8.81 | ND | 8.55 | ND |
| Testis | ND | ND | NA | NA | 9.82 | ND | 9.16 | ND |
| Thymus | ND | ND | NA | NA | 13.9 | ND | 10.7 | 8.16 |
| Tumor | ND | ND | NA | NA | 14.8 | 5.18 | 6.21 | 8.22 |

NA: Not applicable;
ND: not detected;
BLOQ: Below the Limit Of Quantitation

Mouse 107 seemed to clear AMXT 1501 faster than mouse 105. Tumor contained 14.82 and 5.18 μg/g for mice 105 and 107, respectively. No AMXT 1501 was detected in the brain, which can either indicate the absence of brain exposure or an exposure at concentration levels below 3.1 μg/g.

In the combination therapy, lung was again the organ presenting the highest concentration in both animals treated (14.54 and 10.04 μg/g for mice 284 and 286, respectively). Tumor was exposed to 10.0 and 7.21 μg/g, respectively. The combination therapy did not seem to induce significant changes in the AMXT 1501 distribution, and lead to comparable exposure of the tumor. No specific accumulation in tissue was suspected.

The lower limit of detection of AMXT 1501 was 3.1 μg/g or 5.4 μM.

Distribution of DFMO. MSI revealed that DFMO was mainly located in the intestine contents, whether administered alone or in combination. However other tissues were also exposed, as was demonstrated by QMSI.

Concentrations measured in mice treated with DFMO alone ranged from 14.5 to 3487 g/g, from testis to intestine contents, respectively (Table 6.4).

TABLE 6.4
Quantitation of DFMO in μg/g in the whole-body section

| Treatment | Vehicle | | DFMO | | AMXT-1501 | | COMBO | |
|---|---|---|---|---|---|---|---|---|
| Animal ID | 001 | 018 | 49 | 082 | 105 | 107 | 284 | 286 |
| Brain | ND | ND | NA | ND | NA | NA | NA | NA |
| Brown fat | ND | ND | NA | 22.6 | NA | NA | 27.5 | NA |
| Cardiac blood | NC | ND | 27.8 | 16.5 | NA | NA | 35.4 | 20.9 |
| Eye | ND | ND | 33.0 | 28.8 | NA | NA | 92.1 | 49.3 |
| Intestine | ND | ND | 3487 | 2267 | NA | NA | 8702 | 11264 |
| contents | | | | | | | | |
| Intestine wall | ND | ND | 1709 | 803 | NA | NA | NA | 6156 |
| Kidney | ND | ND | 77.6 | 133 | NA | NA | NA | NA |
| Liver | ND | ND | 36.1 | 28.5 | NA | NA | 44.7 | 33.0 |
| Lung | ND | ND | 48.7 | 24.8 | NA | NA | 64.7 | 43.1 |
| Myocardium | ND | ND | 10.0 | ND | NA | NA | 28.5 | 26.2 |
| Pancreas | ND | ND | NA | ND | NA | NA | 83.0 | NA |
| Spleen | ND | ND | NA | ND | NA | NA | 83.6 | NA |
| Stomach | ND | ND | 411 | 34.1 | NA | NA | 111 | 4544 |
| contents | | | | | | | | |
| Stomach wall | ND | ND | 216 | ND | NA | NA | 44.5 | 2036 |
| Testis | ND | ND | 14.5 | ND | NA | NA | NA | 48.2 |
| Thymus | ND | ND | NA | 20.9 | NA | NA | NA | 26.7 |
| Tumor | ND | ND | NA | 35.1 | NA | NA | NA | 30.4 |
| White fat | ND | ND | NA | ND | NA | NA | NA | NA |

NA: Not applicable;
ND: not detected;
BLOQ: Below the Limit Of Quantitation

High concentrations—in the mg/g range—were measured in the intestine contents after oral administration of DFMO. This may be due to the timing of animal sacrifice. Mice drink during dark periods of the day and relatively higher amounts of DFMO containing drinking water are consumed before morning sacrifice. Relatively high concentrations of circulating DFMO are observed in the cardiac blood (16 to 35 μg/g) with comparable concentrations in the combination and single administration regimens. Eye, kidney, liver, lung, testis and thymus are also consistently exposed to DFMO, with little difference between treatments, and concentrations ranging from 14 μg/g (testis) to 92 μg/g (eye). Although hydrophilic, DFMO has also been quantified in brown fat at around 25 μg/g. As regards pancreas and spleen, only mouse 284 (combination therapy) showed a relatively high concentration of DFMO, whereas for mouse 082 (DFMO alone) these organs showed no detection. Tumor, when observable, contained 30 to g/g DFMO. The combination therapy did not seem to induce significant changes in the DFMO distribution, and led to comparable exposure of the tumor. No specific accumulation in tissue was suspected.

The lower limit of detection of DFMO was 2.7 μg/g or 14.8 μM.

Distribution of Polyamine Metabolites. Putrescine was not detected in any group (treatment or vehicle). The low molecular weight of the molecule may explain this analytical challenge, at least for the vehicle group. In the DFMO-treated samples, inhibition of ornithine decarboxylase may also contribute to the lack of detection of putrescine. Based on direct analysis of calibration spots of labeled putrescine, a lower limit of detection of putrescine was higher than 280 μg/g (spot at 100 μM was not detected on control liver section). Previous tumor putrescine levels homogenates in this model were 26.8 μg/g (by LC-MS/MS).

Spermidine and spermine were imaged and quantified, and the results are shown in Tables 6.5 and 6.6, respectively. The lower limit of detection based on the calibration curve using labeled spermidine was 0.80 μg/g, while for spermine it was 1.10 μg/g.

TABLE 6.5
Quantitation of spermidine in μM in the whole-body section

| Treatment | Vehicle | | DFMO | | AMXT-1501 | | COMBO | |
|---|---|---|---|---|---|---|---|---|
| Animal ID | 001 | 018 | 049 | 082 | 105 | 107 | 284 | 286 |
| Blood | NA | NA | ND | NA | NA | NA | NA | ND |
| Brain | ND | NA | 1.86 | NA | ND | NA | NA | NA |
| Brown fat | ND | ND | NA | 3.80 | ND | NA | ND | NA |
| Cardiac blood | ND | ND | 0.00 | 0.00 | ND | NA | ND | ND |
| Eye | ND | 9.14 | 6.11 | 5.24 | ND | 3.21 | 2.93 | 3.03 |
| Intestine contents | 4.80 | 14.95 | 43.17 | 5.61 | 6.36 | 9.59 | 20.39 | 6.00 |
| Intestine wall | 10.43 | 4.07 | 4.56 | ND | NA | NA | NA | 4.90 |
| Kidney | ND | NA | 1.35 | ND | ND | NA | NA | NA |
| Liver | ND | 1.10 | 6.67 | ND | ND | ND | ND | ND |
| Lung | 1.43 | 3.24 | 4.53 | 2.64 | ND | ND | 21.62 | 2.51 |
| Myocardium | NA | ND | ND | NA | ND | NA | ND | ND |
| Pancreas | 8.62 | 6.79 | NA | NA | ND | NA | 20.06 | NA |
| Spleen | 6.27 | 5.74 | NA | NA | ND | NA | NA | NA |
| Stomach contents | ND | ND | ND | ND | 2.05 | 0.00 | ND | ND |
| Stomach wall | NA | 1.16 | 1.13 | NA | NA | NA | ND | 1.59 |
| Testis | NA | 1.43 | 1.97 | NA | ND | NA | ND | 3.53 |
| Thymus | 3.78 | 6.07 | NA | 6.00 | ND | NA | NA | 1.89 |
| Tumor | 5.29 | 0.90 | 5.39 | ND | ND | 1.32 | NA | ND |
| Urogenital tract | NA | NA | NA | NA | NA | NA | 1.09 | NA |
| White fat | NA | NA | NA | NA | 5.26 | NA | NA | NA |

NA: Not applicable;
ND: not detected;
BLOQ: Below the Limit Of Quantitation

TABLE 6.6
Quantitation of spermine in μM in the whole-body section

| Treatment | Vehicle | | DFMO | | AMXT-1501 | | COMBO | |
|---|---|---|---|---|---|---|---|---|
| Animal ID | 001 | 018 | 049 | 082 | 105 | 107 | 284 | 286 |
| Blood | NA | NA | ND | NA | NA | NA | NA | ND |
| Brain | ND | NA | ND | NA | ND | NA | NA | NA |
| Brown fat | ND | ND | NA | ND | ND | NA | ND | NA |
| Cardiac blood | ND | ND | ND | ND | NA | NA | ND | NA |
| Eye | ND | 17.56 | 16.03 | 8.07 | 4.19 | 5.03 | 3.77 | 6.10 |
| Intestine contents | ND | 1.98 | 2.46 | ND | ND | ND | 1.29 | ND |
| Intestine wall | ND | ND | 3.44 | ND | NA | NA | NA | 1.12 |
| Kidney | 2.51 | NA | 4.69 | ND | ND | NA | NA | NA |
| Liver | ND | ND | 2.14 | ND | ND | ND | ND | 1.17 |
| Lung | ND | 2.49 | 4.78 | 7.91 | ND | ND | 14.63 | 4.37 |
| Myocardium | NA | ND | BLOQ | NA | ND | VA | 1.45 | ND |
| Pancreas | ND | 2.28 | NA | NA | ND | NA | 1.14 | NA |
| Spleen | BLOQ | 3.92 | NA | NA | ND | NA | NA | NA |
| Stomach contents | ND | ND | 0.00 | 0.00 | ND | ND | ND | ND |
| Stomach wall | NA | ND | BLOQ | NA | NA | NA | 1.43 | 3.04 |
| Testis | NA | 3.92 | 7.40 | NA | ND | NA | 3.04 | ND |
| Thymas | ND | 2.33 | NA | BLOQ | ND | NA | NA | 3.92 |
| Tumor | ND | ND | 6.03 | ND | ND | ND | NA | 3.82 |
| Urogenital tract | NA | NA | NA | NA | NA | NA | 1.71 | NA |
| White fat | NA | NA | NA | NA | ND | NA | NA | NA |

NA: Not applicable;
ND: not detected;
BLOQ: Below the Limit Of Quantitation

Spermidine and spermine were widely and heterogeneously distributed in most tissues investigated. The most predominant tissues containing spermidine were intestine contents and pancreas, while all other organs have a lower but detectable intensity, including tumor. Myocardium and blood did not show any significant signal. Spermine was also widely distributed, with highest intensities observed in the eye, lung, testis and tumor. Blood did not show any detectable spermine. In addition, spermine does not seem to be distributed in fatty tissues (brain, brown and white fat). Intestine contents showed the highest average amount of spermidine, although with high inter-individual variability (from 4.80 to 43.2 μM).

In the vehicle-treated eyes, spermidine was measured at 9.14 μM, while treatment reduced spermidine levels to 6 μM in DFMO only group and to 3 μM in AMXT 1501 only and combination groups. Similarly, in the intestine wall (when measurable), spermidine levels were lower in treated groups than in vehicle groups.

In contrast, average spermidine levels in the lung were increased, mainly due to the high concentration of 21.6 μM measured in mouse 284. A similar effect was noted in the pancreas, where spermidine was quantified at around 7.7 μM in vehicle and 20.1 μM in one combination treatment animal (284). All other organs presented detectable spermidine concentrations, but below the limit of detection (0.8 μg/g or 5.5 μM).

Spermine was widely distributed (Table 10). The eye contained the highest concentrations (17.6 μM in vehicle and 12 μM on average in DFMO only group, while in AMXT 1501 groups, concentrations fell to around 5 μM). In contrast to spermidine, spermine is barely observed in intestine contents. In the lung, spermine levels are low in vehicle and AMXT 1501 groups and becomes quantifiable only in presence of DFMO (average of 6.35 μM in DFMO group and average of 9.50 μM in combination treatment group). All other organs present detectable concentrations but below the limit of detection (1.1 μg/g or 5.4 μM).

In tumor, the AMXT 1501 only group shows the lowest levels of both spermidine and spermine. Spermidine levels are slightly decreased in the combination treatment group, while spermine levels are increased in the combination group versus AMXT 1501 alone. The highest levels of spermine and spermidine were measured in DFMO group.

Note: many of these observations are extrapolations below the lower limit of quantification. Raw determination of concentrations of spermine and spermidine in tumor are reported in Table 6.7.

TABLE 6.7
Raw determination of concentrations of spermidine and spermine
in μM in tumor

| | Spermidine | | Spermine | |
|---|---|---|---|---|
| | Values | Average | Value | Average |
| Vehicle | 5.29 | 3.09 | BLOD | BLOD |
| | 0.90 | | BLOD | |
| DFMO | 5.39 | 5.39 | 6.03 | 6.03 |
| | BLOD | | BLOD | |
| AMXT - 1501 | BLOD | 1.32 | BLOD | BLOD |
| | 1.32 | | BLOD | |
| COMBO | BLOD | BLOD | 3.82 | 3.82 |

Conclusions. Most organs were exposed to AMXT 1501 and DFMO. However, AMXT 1501 showed a more homogeneous distribution, with concentration ranging from 3.36 to 17.0 μg/g (5.9 to 29.9 μM). DFMO showed a more heterogeneous exposure, and when observed, DFMO showed concentrations from 9.95 μg/g to 11,264 μg/g (54.6 to 61,822 μM), with high exposure of stomach and GI tract. Concentrations of AMXT 1501 in the tumor were 10.0 μg/g (17.6 μM) and 7.21 μg/g (12.7 μM) in AMXT 1501 only and combination treatment groups, respectively. Concentrations of DFMO in the tumor were 35 μg/g (192 μM) and 30 μg/g (165 μM) in DFMO only and combination treatment groups, respectively.

These tumor drug concentrations are both expected to be higher than concentrations required for full engagement of the drugs' respective biological targets. For AMXT 1501, an average 51 nM EC₅₀ concentration is required against growth of four cell lines in culture. For DFMO, a Kᵢ value of 39 μM against its target (ornithine decarboxylase, isolated from rat liver) has been reported. Therefore, in the tumors of combination treatment group mice, a 250 times higher concentration of AMXT 1501, and 4.2 times higher DFMO concentration versus their respective EC₅₀/Kᵢ values were observed. For reference, 51 nM AMXT 1501 is 0.029 μg/g, and 39 μM DFMO is 6.4 μg/g.

While putrescine was been detected, spermidine and spermine were widely distributed. In tumor, the AMXT 1501 only treatment group showed the lowest levels of both spermidine and spermine. In the combination treatment group, spermine levels increased compared to AMXT 1501 alone. The highest levels of spermine and spermidine were measured in DFMO only group.

### REFERENCES

1. Pegg, A. E. & Feith, D. J. Polyamines and neoplastic growth. Biochem Soc Trans 35, 295-299 (2007).
2. Linsalata, M., et al. Prognostic value of tissue polyamine levels in human colorectal carcinoma. Anticancer Res 22, 2465-2469 (2002).
3. O'Brien, T. G. The induction of ornithine decarboxylase as an early, possibly obligatory, event in mouse skin carcinogenesis. Cancer Res 36, 2644-2653 (1976).
4. DiGiovanni, J. Multistage carcinogenesis in mouse skin. Pharmacol Ther 54, 63-128 (1992).
5. O'Brien, T. G., Megosh, L. C., Gilliard, G. & Soler, A. P. Ornithine decarboxylase overexpression is a sufficient condition for tumor promotion in mouse skin. Cancer Res 57, 2630-2637 (1997).
6. Smith, M. K., Trempus, C. S. & Gilmour, S. K. Co-operation between follicular ornithine decarboxylase and v-Ha-ras induces spontaneous papillomas and malignant conversion in transgenic skin. Carcinogenesis 19, 1409-1415 (1998).
7. Chen, Y., Megosh, L. C., Gilmour, S. K., Sawicki, J. A. & O'Brien, T. G. K6/ODC transgenic mice as a sensitive model for carcinogen identification. Toxicol Lett 116, 27-35 (2000).
8. Lan, L., Hayes, C. S., Laury-Kleintop, L. & Gilmour, S. K. Suprabasal induction of ornithine decarboxylase in adult mouse skin is sufficient to activate keratinocytes. J Invest Dermatol 124, 602-614 (2005).
9. Hayes, C. S., et al. A prolonged and exaggerated wound response with elevated ODC activity mimics early tumor development. Carcinogenesis 32, 1340-1348 (2011).
10. Bello-Fernandez, C. & Cleveland, J. L. c-myc transactivates the ornithine decarboxylase gene. Curr Top Microbiol Immunol 182, 445-452 (1992).
11. Nilsson, J. A., et al. Targeting ornithine decarboxylase in Myc-induced lymphomagenesis prevents tumor formation. Cancer Cell 7, 433-444 (2005).
12. Soda, K. The mechanisms by which polyamines accelerate tumor spread. J Exp Clin Cancer Res 30, 95 (2011).
13. Keough, M. P., Hayes, C. S., DeFeo, K. & Gilmour, S. K. Elevated epidermal ornithine decarboxylase activity suppresses contact hypersensitivity. J Invest Dermatol 131, 158-166 (2011).
14. Medina, C. B., et al. Metabolites released from apoptotic cells act as tissue messengers. Nature 580, 130-135 (2020).
15. Gerner, E. W. & Meyskens, F. L., Jr. Polyamines and cancer: old molecules, new understanding. Nat Rev Cancer 4, 781-792 (2004).
16. Chamaillard, L., et al. Polyamine deprivation prevents the development of tumour-induced immune suppression. Br J Cancer 76, 365-370 (1997).
17. Persson, L., Holm, I., Ask, A. & Heby, O. Curative effect of DL-2-difluoromethylornithine on mice bearing mutant L1210 leukemia cells deficient in polyamine uptake. Cancer Res 48, 4807-4811 (1988).
18. Burns, M. R., Graminski, G. F., Weeks, R. S., Chen, Y. & O'Brien, T. G. Lipophilic lysine-spermine conjugates are potent polyamine transport inhibitors for use in combination with a polyamine biosynthesis inhibitor. J Med Chem 52, 1983-1993 (2009).
19. Hayes, C. S., Burns, M. R. & Gilmour, S. K. Polyamine blockade promotes antitumor immunity. Oncoimmunology 3, e27360 (2014).
20. Hayes, C. S., et al. Polyamine-blocking therapy reverses immunosuppression in the tumor microenvironment. Cancer Immunol Res 2, 274-285 (2014).
21. Burns, M. R., et al. Abstract 2006: AMX-513 polyamine depletion therapy inhibits tumor growth and reverses immunosuppression in cancers including MYC-driven neuroblastoma and pancreatic cancer. Cancer Research 77, 2006-2006 (2017).
22. Gamble, L. D., et al. Inhibition of polyamine synthesis and uptake reduces tumor progression and prolongs survival in mouse models of neuroblastoma. Sci Transl Med 11(2019).
23. Khan, A., et al. Dual targeting of polyamine synthesis and uptake in diffuse intrinsic pontine gliomas. Nat Commun 12, 971 (2021).
24. Seiler, N., Delcros, J. G. & Moulinoux, J. P. Polyamine transport in mammalian cells. An update. Int J Biochem Cell Biol 28, 843-861 (1996).
25. Metcalf, B. W., et al. Catalytic irreversible inhibition of mammalian ornithine decarboxylase (E. C.4.1.1.17) by substrate and product analogs. Journal of the American Chemical Society 100, 2551-2553 (1978).
26. Chamaillard, L., Quemener, V., Havouis, R. & Moulinoux, J. P. Polyamine deprivation stimulates natural killer cell activity in cancerous mice. Anticancer Res 13, 1027-1033 (1993).
27. Ask, A., Persson, L. & Heby, O. Increased survival of L1210 leukemic mice by prevention of the utilization of extracellular polyamines. Studies using a polyamine-uptake mutant, antibiotics and a polyamine-deficient diet. Cancer Lett 66, 29-34 (1992).
28. Sarhan, S., Knodgen, B. & Seiler, N. The gastrointestinal tract as polyamine source for tumor growth. Anticancer Res 9, 215-223 (1989).
29. Samal, K., et al. AMXT-1501, a novel polyamine transport inhibitor, synergizes with DFMO in inhibiting neuroblastoma cell proliferation by targeting both ornithine decarboxylase and polyamine transport. Int J Cancer 133, 1323-1333 (2013).
30. Misquith, A., et al. In vitro evaluation of TLR4 agonist activity: formulation effects. Colloids Surf B Biointerfaces 113, 312-319 (2014).
31. Zaitseva, M., et al. Use of human MonoMac6 cells for development of in vitro assay predictive of adjuvant safety in vivo. Vaccine 30, 4859-4865 (2012).
32. Bowlin, T. L., McKown, B. J., Davis, G. F. & Sunkara, P. S. Effect of polyamine depletion in vivo by DL-alpha-difluoromethylornithine on functionally distinct populations of tumoricidal effector cells in normal and tumor-bearing mice. Cancer Res 46, 5494-5498 (1986).
33. Kohl, N. E., Gee, C. E. & Alt, F. W. Activated expression of the N-myc gene in human neuroblastomas and related tumors. Science 226, 1335-1337 (1984).
34. Geerts, D., et al. The polyamine metabolism genes ornithine decarboxylase and antizyme 2 predict aggressive behavior in neuroblastomas with and without MYCN amplification. Int J Cancer 126, 2012-2024 (2010).
35. Evageliou, N. F., et al. Polyamine Antagonist Therapies Inhibit Neuroblastoma Initiation and Progression. Clin Cancer Res 22, 4391-4404 (2016).
36. Weiss, W. A., Aldape, K., Mohapatra, G., Feuerstein, B. G. & Bishop, J. M. Targeted expression of MYCN causes neuroblastoma in transgenic mice. EMBO J 16, 2985-2995 (1997).
37. Eriksson, C., Masaki, N., Yao, I., Hayasaka, T. & Setou, M. MALDI Imaging Mass Spectrometry-A Mini Review of Methods and Recent Developments. Mass Spectrom (Tokyo) 2, 50022 (2013).
38. Romijn, J. C., Verkoelen, C. F. & Splinter, T. A. Problems of pharmacokinetic studies on alpha-difluoromethylornithine in mice. Cancer Chemother Pharmacol 19, 30-34 (1987).
39. Kano, Y., et al. Increased blood spermine levels decrease the cytotoxic activity of lymphokine-activated killer cells: a novel mechanism of cancer evasion. Cancer Immunol Immunother 56, 771-781 (2007).
40. Bowlin, T. L., Hoeper, B. J., Rosenberger, A. L., Davis, G. F. & Sunkara, P. S. Effects of three irreversible inhibitors of ornithine decarboxylase on macrophage-mediated tumoricidal activity and antitumor activity in B16F1 tumor-bearing mice. Cancer Res 50, 4510-4514 (1990).
41. Flescher, E., Bowlin, T. L. & Talal, N. Polyamine oxidation down-regulates IL-2 production by human peripheral blood mononuclear cells. J Immunol 142, 907-912 (1989).
42. Gensler, H. L. Prevention by alpha-difluoromethylornithine of skin carcinogenesis and immunosuppression induced by ultraviolet irradiation. J Cancer Res Clin Oncol 117, 345-350 (1991).
43. Zhang, M., et al. Spermine inhibits proinflammatory cytokine synthesis in human mononuclear cells: a counterregulatory mechanism that restrains the immune response. J Exp Med 185, 1759-1768 (1997).
44. Wolff, A. C., et al. A Phase II study of the polyamine analog N1,N11-diethylnorspermine (DENSpm) daily for five days every 21 days in patients with previously treated metastatic breast cancer. Clin Cancer Res 9, 5922-5928 (2003).
45. Hahm, H. A., et al. Phase I study of N(1),N(11)-diethylnorspermine in patients with non-small cell lung cancer. Clin Cancer Res 8, 684-690 (2002).
46. Wilding, G., et al. Phase I trial of the polyamine analog N1,N14-diethylhomospermine (DEHSPM) in patients with advanced solid tumors. Invest New Drugs 22, 131-138 (2004).
47. Herbst, R. S., et al. A phase I/IA trial of continuous five-day infusion of squalamine lactate (MSI-1256F) plus carboplatin and paclitaxel in patients with advanced non-small cell lung cancer. Clin Cancer Res 9, 4108-4115 (2003).
48. Bhargava, P., et al. A phase I and pharmacokinetic study of squalamine, a novel antiangiogenic agent, in patients with advanced cancers. Clin Cancer Res 7, 3912-3919 (2001).
49. Dhingra, K., et al. Phase I clinical and pharmacological study of suppression of human antimouse antibody response to monoclonal antibody L6 by deoxyspergualin. Cancer Res 55, 3060-3067 (1995).
50. Kruczynski, A., et al. F14512, a polyamine-vectorized anti-cancer drug, currently in clinical trials exhibits a marked preclinical anti-leukemic activity. Leukemia 27, 2139-2148 (2013).
51. Tierny, D., et al. Phase I Clinical Pharmacology Study of F14512, a New Polyamine-Vectorized Anticancer Drug, in Naturally Occurring Canine Lymphoma. Clin Cancer Res 21, 5314-5323 (2015).
52. De Clercq, E. Recent advances on the use of the CXCR4 antagonist plerixafor (AMD3100, Mozobil) and potential of other CXCR4 antagonists as stem cell mobilizers. Pharmacol Ther 128, 509-518 (2010).
53. Lack, N. A., et al. A pharmacokinetic-pharmacodynamic model for the mobilization of CD34+ hematopoietic progenitor cells by AMD3100. Clin Pharmacol Ther 77, 427-436 (2005).
54. Lu, J. Triethylenetetramine pharmacology and its clinical applications. Mol Cancer Ther 9, 2458-2467 (2010).
55. Scalabrino, G. & Ferioli, M. E. Polyamines in mammalian tumors. Part II. Adv Cancer Res 36, 1-102 (1982).
56. Alberts, D. S., et al. Chemoprevention of human actinic keratoses by topical 2-(difluoromethyl)-dl-ornithine. Cancer Epidemiol Biomarkers Prev 9, 1281-1286 (2000).
57. Marton, L. J., et al. The relationship of polyamines in cerebrospinal fluid to the presence of central nervous system tumors. Cancer Res 36, 973-977 (1976).
58. Pan, X., et al. Cerebrospinal Fluid Spermidine, Glutamine and Putrescine Predict Postoperative Delirium Following Elective Orthopaedic Surgery. Sci Rep 9, 4191 (2019).
59. Siu, L. L., et al. A phase I and pharmacokinetic study of SAM486A, a novel polyamine biosynthesis inhibitor, administered on a daily-times-five every-three-week schedule in patients with Advanced solid malignancies. Clin Cancer Res 8, 2157-2166 (2002).
60. Canizares, F., et al. Prognostic value of ornithine decarboxylase and polyamines in human breast cancer: correlation with clinicopathologic parameters. Clin Cancer Res 5, 2035-2041 (1999).
61. Hixson, L. J., et al. Ornithine decarboxylase and polyamines in colorectal neoplasia and mucosa. Cancer Epidemiol Biomarkers Prev 2, 369-374 (1993).
62. Gerner, E. W., Garewal, H. S., Emerson, S. S. & Sampliner, R. E. Gastrointestinal tissue polyamine contents of patients with Barrett's esophagus treated with alpha-difluoromethylornithine. Cancer Epidemiol Biomarkers Prev 3, 325-330 (1994).
63. Boyle, J. O., Meyskens, F. L., Jr., Garewal, H. S. & Gerner, E. W. Polyamine contents in rectal and buccal mucosae in humans treated with oral difluoromethylornithine. Cancer Epidemiol Biomarkers Prev 1, 131-135 (1992).
64. Mitchell, M. F., et al. Polyamine measurements in the uterine cervix. J Cell Biochem Suppl 28-29, 125-132 (1997).
65. Dimery, I. W., et al. Polyamine metabolism in carcinoma of the oral cavity compared with adjacent and normal oral mucosa. Am J Surg 154, 429-433 (1987).
66. Smithson, D. C., Shelat, A. A., Baldwin, J., Phillips, M. A. & Guy, R. K. Optimization of a non-radioactive high-throughput assay for decarboxylase enzymes. Assay Drug Dev Technol 8, 175-185 (2010).
67. Nilam, M., et al. A Label-Free Continuous Fluorescence-Based Assay for Monitoring Ornithine Decarboxylase Activity with a Synthetic Putrescine Receptor. SLAS Discov 22, 906-914 (2017).
68. Tao, L., et al. CHAF1A Blocks Neuronal Differentiation and Promotes Neuroblastoma Oncogenesis via Metabolic Reprogramming. Adv Sci (Weinh), e2005047 (2021).
69. Garewal, H. S., Sloan, D., Sampliner, R. E. & Fennerty, B. Ornithine decarboxylase assay in human colorectal mucosa. Methodologic issues of importance to quality control. Int J Cancer 52, 355-358 (1992).
70. Danzin, C. & Persson, L. L-ornithine-induced inactivation of mammalian ornithine decarboxylase in vitro. Eur J Biochem 166, 45-48 (1987).
71. Janne, J. & Williams-Ashman, H. G. On the purification of L-ornithine decarboxylase from rat prostate and effects of thiol compounds on the enzyme. J Biol Chem 246, 1725-1732 (1971).
72. Hyv6nen, M. T., Keinänen, T. & Alhonen, L. Assay of Ornithine Decarboxylase and Spermidine/Spermine N1-acetyltransferase Activities. Bio-protocol 4, e1301 (2014).
73. Gaines, D. W., Friedman, L. & McCann, P. P. Apparent ornithine decarboxylase activity, measured by 14CO2 trapping, after frozen storage of rat tissue and rat tissue supernatants. Anal Biochem 174, 88-96 (1988).
74. Janne, J. & Williams-Ashman, H. G. Mammalian ornithine decarboxylase: activation and alteration of physical behaviour by thiol compounds. Biochem J 119, 595-597 (1970).
75. Kilpelainen, P. T. & Hietala, O. A. Activation of rat brain ornithine decarboxylase by GTP. Biochem J 300 (Pt 2), 577-582 (1994).
76. Zawia, N. H., Mattia, C. J. & Bondy, S. C. Differential effects of difluoromethylornithine on basal and induced activity of cerebral ornithine decarboxylase and mRNA. Neuropharmacology 30, 337-343 (1991).
77. O'Brien, T. G., Hietala, O., O'Donnell, K. & Holmes, M. Activation of mouse epidermal tumor ornithine decarboxylase by GTP: evidence for different catalytic forms of the enzyme. Proc Natl Acad Sci USA 84, 8927-8931 (1987).
78. Scalabrino, G., et al. Levels of activity of the polyamine biosynthetic decarboxylases as indicators of degree of malignancy of human cutaneous epitheliomas. J Invest Dermatol 74, 122-124 (1980).
79. Herrera-Ornelas, L., et al. A comparison of ornithine decarboxylase and S-adenosylmethionine decarboxylase activity in human large bowel mucosa, polyps, and colorectal adenocarcinoma. J Surg Res 42, 56-60 (1987).
80. LaMuraglia, G. M., Lacaine, F. & Malt, R. A. High ornithine decarboxylase activity and polyamine levels in human colorectal neoplasia. Ann Surg 204, 89-93 (1986).
81. Manni, A., Mauger, D., Gimotty, P. & Badger, B. Prognostic influence on survival of increased ornithine decarboxylase activity in human breast cancer. Clin Cancer Res 2, 1901-1906 (1996).
82. Bukin, Y. V., et al. Effect of prolonged beta-carotene or DL-alpha-tocopheryl acetate supplementation on ornithine decarboxylase activity in human atrophic stomach mucosa. Cancer Epidemiol Biomarkers Prev 4, 865-870 (1995).
83. Dyshlovoy, S. A., et al. Proteomic profiling of germ cell cancer cells treated with aaptamine, a marine alkaloid with antiproliferative activity. J Proteome Res 11, 2316-2330 (2012).
84. Klier, H., et al. Isolation and structural characterization of different isoforms of the hypusine-containing protein eIF-5A from HeLa cells. Biochemistry 34, 14693-14702 (1995).
85. Poulin, R., Lu, L., Ackermann, B., Bey, P. & Pegg, A. E. Mechanism of the irreversible inactivation of mouse ornithine decarboxylase by alpha-difluoromethylornithine. Characterization of sequences at the inhibitor and coenzyme binding sites. J Biol Chem 267, 150-158 (1992).
86. Fujimura, K., Wang, H., Watson, F. & Klemke, R. L. KRAS Oncoprotein Expression Is Regulated by a Self-Governing eIF5A-PEAK1 Feed-Forward Regulatory Loop. Cancer Res 78, 1444-1456 (2018).
87. Gobert, A. P., et al. Hypusination Orchestrates the Antimicrobial Response of Macrophages. Cell Rep 33, 108510 (2020).
88. Puleston, D. J., et al. Polyamines and eIF5A Hypusination Modulate Mitochondrial Respiration and Macrophage Activation. Cell Metab 30, 352-363 e358 (2019).
89. Nishiki, Y., et al. Characterization of a novel polyclonal anti-hypusine antibody. Springerplus 2, 421 (2013).
90. Coni, S., et al. Blockade of EIF5A hypusination limits colorectal cancer growth by inhibiting MYC elongation. Cell Death Dis 11, 1045 (2020).
91. Zhai, Q., et al. Structural Analysis and Optimization of Context-Independent Anti-Hypusine Antibodies. J Mol Biol 428, 603-617 (2016).
92. Ray, R. M., Bhattacharya, S., Bavaria, M. N., Viar, M. J. & Johnson, L. R. Spermidine, a sensor for antizyme 1 expression regulates intracellular polyamine homeostasis. Amino Acids 46, 2005-2013 (2014).
93. Ishiwata, K., Abe, Y., Matsuzawa, T. & Ido, T. Tumor uptake studies of D,L-[5-14C]ornithine and D,L-2-difluoromethyl [5-14C]ornithine. Int J Rad Appl Instrum B 15, 119-122 (1988).
94. Levin, V. A., Csejtey, J. & Byrd, D. J. Brain, CSF, and tumor pharmacokinetics of alpha-difluoromethylornithine in rats and dogs. Cancer Chemother Pharmacol 10, 196-199 (1983).
95. Pendyala, L., Creaven, P. J. & Porter, C. W. Urinary and erythrocyte polyamines during the evaluation of oral alpha-difluoromethylornithine in a phase I chemoprevention clinical trial. Cancer Epidemiol Biomarkers Prev 2, 235-241 (1993).
96. Griffin, C. A., et al. Phase I trial and pharmacokinetic study of intravenous and oral alpha-difluoromethylornithine. Invest New Drugs 5, 177-186 (1987).
97. Messing, E. M., et al. Low-dose difluoromethylornithine and polyamine levels in human prostate tissue. J Natl Cancer Inst 91, 1416-1417 (1999).
98. Carbone, P. P., et al. Phase I chemoprevention study of difluoromethylornithine in subjects with organ transplants. Cancer Epidemiol Biomarkers Prev 10, 657-661 (2001).
99. Meyskens, F. L., Jr., et al. Dose de-escalation chemoprevention trial of alpha-difluoromethylornithine in patients with colon polyps. J Natl Cancer Inst 86, 1122-1130 (1994).
100. Meyskens, F. L., Jr., et al. Effect of alpha-difluoromethylornithine on rectal mucosal levels of polyamines in a randomized, double-blinded trial for colon cancer prevention. J Natl Cancer Inst 90, 1212-1218 (1998).
101. Meyskens, F. L., Jr., et al. Difluoromethylornithine plus sulindac for the prevention of sporadic colorectal adenomas: a randomized placebo-controlled, double-blind trial. Cancer Prev Res (Phila) 1, 32-38 (2008).
102. Thompson, P. A., et al. Levels of rectal mucosal polyamines and prostaglandin E2 predict ability of DFMO and sulindac to prevent colorectal adenoma. Gastroenterology 139, 797-805, 805 e791 (2010).
103. Liao, X., Liang, W., Wiedmann, T., Wattenberg, L. & Dahl, A. Lung distribution of the chemopreventive agent difluoromethylornithine (DFMO) following oral and inhalation delivery. Exp Lung Res 30, 755-769 (2004).
104. Luk, G. D., Goodwin, G., Marton, L. J. & Baylin, S. B. Polyamines are necessary for the survival of human small-cell lung carcinoma in culture. Proc Natl Acad Sci USA 78, 2355-2358 (1981).
105. Loiseau, P. M., Czok, M., Chauffert, O., Bourass, J. & Letourneux, Y. Studies on lipidomimetic derivatives of alpha-difluoromethylornithine (DFMO) to enhance the bioavailability in a Trypanosoma B. brucei murine trypanosomiasis model. Parasite 5, 239-246 (1998).
106. Suli-Vargha, H., et al. In vitro cytotoxic effect of difluoromethylomithine increased nonspecifically by peptide coupling. J Pharm Sci 86, 997-1000 (1997).
107. Claverie, N. & Mamont, P. S. Comparative antitumor properties in rodents of irreversible inhibitors of L-ornithine decarboxylase, used as such or as prodrugs. Cancer Res 49, 4466-4471 (1989).
108. Bitonti, A. J., Bacchi, C. J., McCann, P. P. & Sjoerdsma, A. Uptake of alpha-difluoromethylornithine by Trypanosoma brucei brucei. Biochem Pharmacol 35, 351-354 (1986).
109. Erwin, B. G. & Pegg, A. E. Uptake of alpha-difluoromethylornithine by mouse fibroblasts. Biochem Pharmacol 31, 2820-2823 (1982).
110. Vig, B. S., Huttunen, K. M., Laine, K. & Rautio, J. Amino acids as promoieties in prodrug design and development. Adv Drug Deliv Rev 65, 1370-1385 (2013).
111. Zhang, L., Sui, C., Yang, W. & Luo, Q. Amino acid transporters: Emerging roles in drug delivery for tumor-targeting therapy. Asian J Pharm Sci 15, 192-206 (2020).
112. Slocum, R. D., Bitonti, A. J., McCann, P. P. & Feirer, R. P. DL-alpha-difluoromethyl[3,4-3H]arginine metabolism in tobacco and mammalian cells. Inhibition of ornithine decarboxylase activity after arginase-mediated hydrolysis of DL-alpha-difluoromethylarginine to DL-alpha-difluoromethylornithine. Biochem J 255, 197-202 (1988).
113. Liu, L., et al. Polyamine-modulated expression of c-myc plays a critical role in stimulation of normal intestinal epithelial cell proliferation. Am J Physiol Cell Physiol 288, C89-99 (2005).
114. Haegele, K. D., Alken, R. G., Grove, J., Schechter, P. J. & Koch-Weser, J. Kinetics of alpha-difluoromethylornithine: an irreversible inhibitor of ornithine decarboxylase. Clin Pharmacol Ther 30, 210-217 (1981).
115. Baker, N., Alsford, S. & Horn, D. Genome-wide RNAi screens in African trypanosomes identify the nifurtimox activator NTR and the eflornithine transporter AAT6. Mol Biochem Parasitol 176, 55-57 (2011).
116. Davis, R. H., Lieu, P. & Ristow, J. L. Neurospora mutants affecting polyamine-dependent processes and basic amino acid transport mutants resistant to the polyamine inhibitor, alpha-difluoromethylornithine. Genetics 138, 649-655 (1994).
117. Ajani, J. A., et al. Evaluation of continuous-infusion alpha-difluoromethylornithine therapy for colorectal carcinoma. Cancer Chemother Pharmacol 26, 223-226 (1990).
118. Ajani, J. A., Ota, D. M., Grossie, V. B., Jr., Levin, B. & Nishioka, K. Alterations in polyamine metabolism during continuous intravenous infusion of alpha-difluoromethylornithine showing correlation of thrombocytopenia with alpha-difluoromethylornithine plasma levels. Cancer Res 49, 5761-5765 (1989).
119. Maddox, A. M., Keating, M. J., McCredie, K. E., Estey, E. & Freireich, E. J. Phase I evaluation of intravenous difluoromethylornithine—a polyamine inhibitor. Invest New Drugs 3, 287-292 (1985).
120. Maddox, A. M., Freireich, E. J., Keating, M. J., Frasier-Scott, K. F. & Haddox, M. K. Alterations in human circulating and bone marrow mononuclear cell polyamine levels in hematologic malignancies as a consequence of difluoromethylornithine administration. Invest New Drugs 6, 125-134 (1988).
121. Milord, F., Loko, L., Ethier, L., Mpia, B. & Pepin, J. Eflornithine concentrations in serum and cerebrospinal fluid of 63 patients treated for Trypanosoma brucei gambiense sleeping sickness. Trans R Soc Trop Med Hyg 87, 473-477 (1993).
122. Na-Bangchang, K., et al. The pharmacokinetics of eflornithine (alpha-difluoromethylornithine) in patients with late-stage T.b. gambiense sleeping sickness. Eur J Clin Pharmacol 60, 269-278 (2004).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of treating cancer in a subject in need thereof, comprising administering to the subject a therapeutically effective amount of AMXT 1501, or a pharmaceutically acceptable salt thereof, and difluoromethylornithine (DFMO), or a pharmaceutically acceptable salt thereof, wherein:
the AMXT 1501, or a pharmaceutically acceptable salt thereof, is administered orally; and
the DFMO, or a pharmaceutically acceptable salt thereof, is administered intravenously.

2. The method of claim 1, wherein the subject is a human.

3. (canceled)

4. (canceled)

5. The method of claim 2, wherein the subject is an adult human or a human aged 12- to 17-years old.

6. The method of claim 1, wherein the AMXT 1501, or a pharmaceutically acceptable salt thereof, is AMXT 1501 dicaprate.

7. The method of claim 6, wherein the AMXT 1501 dicaprate has the following structure:

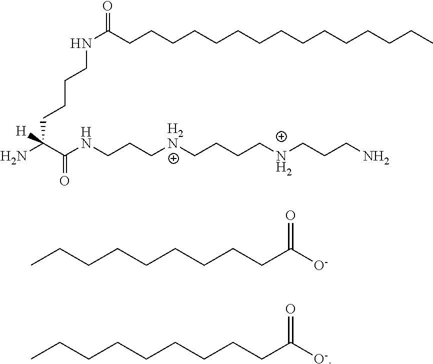

8. The method of claim 1, wherein the AMXT 1501, or a pharmaceutically acceptable salt thereof, is administered twice daily.

9. (canceled)

10. The method of claim 1, wherein the AMXT 1501, or a pharmaceutically acceptable salt thereof, is administered to the subject at a daily dose of from about 100 mg to about 2,500 mg, based on the free base of AMXT 1501.

11. The method of claim 10, wherein the AMXT 1501, or a pharmaceutically acceptable salt thereof, is administered to the subject at a daily dose of about 1,200 mg, based on the free base of AMXT 1501.

12. The method of claim 11, wherein the daily dose is split BID.

13. (canceled)

14. The method of claim 1, wherein the AMXT 1501, or a pharmaceutically acceptable salt thereof, is administered in an enterically-coated, solid oral dosage form.

15. (canceled)

16. (canceled)

17. (canceled)

18. (canceled)

19. The method of claim 1, wherein the DFMO, or a pharmaceutically acceptable salt thereof, is administered to the subject at a daily dose of from about 0.5 g/m² to about 10 g/m², based on the free base of DFMO.

20. The method of claim 19, wherein the DFMO, or a pharmaceutically acceptable salt thereof, is administered to the subject at a daily dose of from about 1 g/m² to about 5 g/m², based on the free base of DFMO.

21. The method of claim 1, wherein the DFMO, or a pharmaceutically acceptable salt thereof, is administered by continuous intravenous infusion.

22. The method of claim 1, wherein the DFMO, or a pharmaceutically acceptable salt thereof, is administered via an ambulatory infusion system.

23. The method of claim 1, wherein the cancer comprises a solid tumor.

24. The method of claim 1, wherein the cancer is skin cancer, melanoma, colon cancer, colorectal cancer, breast cancer, pancreatic cancer, neuroblastoma or diffuse intrinsic pontine glioma (DIPG).

25. The method of claim 1, wherein the cancer is ovarian cancer, thyroid cancer, head and neck cancer, gastric cancer, lung cancer, mesothelioma, esophageal cancer, endometrial cancer, cervical cancer, melanoma, DIPG, colorectal cancer or breast cancer.

26. The method of claim 1, wherein the cancer is a pediatric cancer.

27. The method of claim 1, further comprising administering one or more additional therapeutically active agents to the subject.

28. A method of treating cancer in a subject in need thereof, comprising administering to the subject a therapeutically effective amount of AMXT 1501, or a pharmaceutically acceptable salt thereof, and difluoromethylornithine (DFMO), or a pharmaceutically acceptable salt thereof,
wherein the AMXT 1501, or a pharmaceutically acceptable salt thereof, is administered orally QD or BID at a daily dose of greater than 80 mg and less than 2,500 mg, based on the free base of AMXT 1501.

29. (canceled)

30. (canceled)
